(19) United States
(12) Patent Application Publication
(10) Pub. No.: US 2010/0009931 A1
(43) Pub. Date: Jan. 14, 2010

(54) ASSOCIATION OF A TENSOR AGENT OR DEVICE AND A SACCHARIDE COMPOUND

(75) Inventors: Julien Laboureau, Paris (FR); Guillaume Cassin, Villebon Sur Yvette (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/299,563

(22) PCT Filed: May 4, 2007
(86) PCT No.: PCT/FR07/51222

§ 371 Date: Jun. 29, 2009

Related U.S. Application Data
(60) Provisional application No. 60/800,409, filed on May 16, 2006.
(60) Provisional application No. 60/800,410, filed on May 16, 2006.

(30) Foreign Application Priority Data
May 5, 2006 (FR) 0651644
May 5, 2006 (FR) 0651645

(51) Int. Cl. A61K 8/73; A61K 8/60; A61Q 19/00

(52) U.S. Cl. 514/54; 514/23

(57) ABSTRACT

The invention concerns a composition for topical application on the skin comprising, in a physiological acceptable medium, at least one saccharide compound which augments the expression of mechanoreceptors in skin cells and at least one tensor agent. The invention also concerns a skin care health kit comprising (a) a composition comprising at least one tensor agent and (b) a composition comprising at least one saccharide compound which increases the expression of mechanoreceptors in skin cells. The invention also concerns a kit comprising (a) a composition comprising at least one saccharide compound which augments the expression of mechanoreceptors in skin cells and (b) a tensor device designed to apply and/or maintain in a controlled manner mechanical constraints on the skin. The invention also concerns a process for cosmetic treatment of the skin comprising the simultaneous or sequential application of said compositions and/or tensor devices.

The present invention relates to the skincare field and is in particular directed towards improving the appearance of facial and/or body skin.

The invention relates in particular to a composition for topical application to the skin comprising, in a physiologically acceptable medium, at least one saccharide compound which increases the expression of mechanoreceptors in skin cells and at least one tensing agent.

The invention also relates to a skincare kit comprising (a) a first composition comprising at least one tensing agent and (b) a second composition comprising a saccharide compound which increases the expression of mechanoreceptors in skin cells.

The invention also relates to a skincare kit comprising (a) a composition comprising a saccharide compound which increases the expression of mechanoreceptors in skin cells and (b) a tensing device intended to apply and/or maintain, in a controlled manner, mechanical stresses to and/or on the skin.

The invention also relates to a process for the cosmetic treatment of the skin, comprising the simultaneous or sequential application of said compositions, aimed in particular at smoothing out wrinkles and fine lines and/or improving the firmness and/or the elasticity of the skin.

The present invention relates in particular to a cosmetic skincare process, comprising the simultaneous or sequential application:
(i) of a composition comprising at least one saccharide compound which increases the expression of mechanoreceptors in skin cells;
(ii) of a tensing device intended to apply and/or maintain, in a controlled manner, mechanical stresses to and/or on the skin.

Said process is aimed in particular at smoothing out wrinkles and fine lines and/or improving the firmness and/or the elasticity of the skin.

The invention also relates to the cosmetic use, in a composition comprising a physiologically acceptable medium, of at least one saccharide compound which increases the expression of mechanoreceptors in skin cells, in combination with a tensing agent, for promoting improved skin homeostasis, increased skin thickness, an improvement in the radiance of the complexion, skin density, regeneration and/or reorganization of the papillary dermis, regeneration and/or reorganization of the extracellular matrix and/or an improvement in the firmness, the elasticity and/or the tonicity of the skin.

The invention also relates to the cosmetic use, in a composition comprising a physiologically acceptable medium, of at least one saccharide compound which increases the expression of mechanoreceptors in skin cells, said composition being combined with a tensing device, for promoting improved skin homeostasis, increased skin thickness, an improvement in the radiance of the complexion, skin density, regeneration and/or reorganization of the papillary dermis, regeneration and/or reorganization of the extracellular matrix and/or an improvement in the firmness, the elasticity and/or the tonicity of the skin.

The invention also relates to the cosmetic use of a composition comprising, in a physiologically acceptable medium, at least one saccharide compound which increases the expression of mechanoreceptors in skin cells, in combination with a tensing agent, for promoting improved skin homeostasis, increased skin thickness, an improvement in the radiance of the complexion, skin density, regeneration and/or reorganization of the papillary dermis, regeneration and/or reorganization of the extracellular matrix and/or an improvement in the firmness, the elasticity and/or the tonicity of the skin.

The invention also relates to the cosmetic use of a kit comprising (a) a composition comprising, in a physiologically acceptable medium, at least one saccharide compound which increases the expression of mechanoreceptors in skin cells and (b) a tensing device, for promoting improved skin homeostasis, increased skin thickness, an improvement in the radiance of the complexion, skin density, regeneration and/or reorganization of the papillary dermis, regeneration and/or reorganization of the extracellular matrix and/or an improvement in the firmness, the elasticity and/or the tonicity of the skin.

The invention is in particular directed towards the treatment of the skin of the face and/or neck. However, the compositions of the invention can also be applied to areas of the body which show a loss of elasticity and/or of firmness, such as the stomach and the thighs.

The skin constitutes a physical barrier between the organism and its environment. It is composed of two tissues: the epidermis and the dermis.

The epidermis is a multistratified keratinizing epithelium which undergoes continual renewal. Keratinocytes constitute the main epidermal cell population and are responsible for maintaining the epithelial structure and for its barrier function. The epidermis rests on an acellular basal membrane, called the dermal-epidermal junction, which ensures cohesion with the dermis.

The epidermis is composed of several strata of cells, the deepest of which is the basal stratum, which is composed of undifferentiated cells. Over time, these cells will differentiate and migrate towards the surface of the epidermis, thereby making up the various strata thereof, until, at the surface of the epidermis, they form the corneocytes, which are dead cells that are removed by desquamation. This surface loss is compensated for by the migration of cells from the basal stratum towards the surface of the epidermis. This is a process of continuous renewal of the skin.

The dermis is an elastic and compressible supporting connective tissue of mesodermal origin and is made up mainly of fibroblasts and an extracellular matrix consisting of fibrous proteins (collagens and elastin) and non-fibrous proteins (proteoglycans and glycoproteins). The dermis is a feeder tissue for the epidermis, but it also plays a fundamental role in the development and growth of the epidermis, and in the differentiation thereof. The fibroblasts and the extracellular matrix also influence the mechanical properties of the skin, in particular its elasticity, its tonicity and its firmness. The fibroblasts and the extracellular matrix also influence the density of the skin.

The homeostasis of the skin, and in particular of the epidermis, is the result of a finely regulated balance between the processes of skin cell proliferation and differentiation. These proliferation and differentiation processes are perfectly regulated: they participate in the renewal and/or in the regeneration of the skin and lead to the maintenance of a constant skin thickness, and in particular a constant epidermal thickness. This homeostasis of the skin also plays a role in maintaining the mechanical properties of the skin, in particular its firmness, tonicity and/or elasticity.

However, this homeostasis of the skin can be impaired by certain physiological factors (age, menopause, hormones, etc.) or environmental factors (UV stress, pollution, oxidative stress, irritant stress, etc.). The regenerative potential of the epidermis becomes less great: the cells of the basal layer divide less actively, which leads in particular to a slowing down and/or a decrease in epidermal renewal. Consequently, cell renewal no longer compensates for the loss of the cells removed at the surface, leading to atrophy of the epidermis and/or to a decrease in skin thickness and/or a loss of elasticity and/or of tonicity and/or of firmness of the skin and/or the formation of wrinkles or fine lines.

These clinical signs are visible on the skin of the face and/or neck, but also on the skin of the body, in particular in areas which show a loss of firmness and/or elasticity, such as the stomach and/or the thighs.

This phenomenon may be accentuated by the menopause: women complain of their skin tightening and becoming dry, or even of the appearance of xerosis. The hormonal deficiencies associated with the menopause are accompanied in particular by a drop in metabolic activity, which could result in a decrease in keratinocyte proliferation and an increase in epidermal differentiation.

The need is understood, therefore, to have agents capable of promoting skin homeostasis in order to maintain and/or increase the thickness of the skin, in particular the skin of the face and/or neck, and thus in particular to smooth out wrinkles and fine lines and/or maintain and/or improve the mechanical properties of the skin, in particular of the skin of the face and/or neck, in particular the firmness, the elasticity and/or the tonicity of the skin.

The prior art discloses the use of soluble cosmetic agents for promoting cell renewal. Mention may, for example, be made of retinoic acid derivatives, and in particular retinol, also known as vitamin A, and esterified derivatives of retinol, which have the effect of promoting keratinocyte proliferation and of inhibiting keratinocyte differentiation, thus making it possible to stimulate epidermal renewal, and to maintain and/or increase epidermal thickness.

The soluble cosmetic agents act conventionally via binding to a receptor which initiates intracellular responses, leading to a regulation of the expression of proteins involved in the processes of epidermal proliferation and/or differentiation. This is termed a direct “biological” effect.

The Applicant has now shown, surprisingly and unexpectedly, that an improvement in skin homeostasis can be obtained via a biomechanical effect provided by the topical application of an effective amount of cosmetic agents, in particular tensing agents.

The Applicant has in fact shown that the topical application of an effective amount of tensing agents, such as acrylic copolymers, to a model of reconstructed skin has the effect of modulating the expression of proteins involved in skin homeostasis.

According to an alternative, the Applicant proposes to use as a replacement for and/or as a supplement to a tensing agent, a tensing device intended to apply, in a controlled manner, mechanical stresses on the skin.

According to the invention, the term “biomechanical effect” is intended to mean the ability of a cosmetic agent, and in particular of a tensing agent, to induce a biological response in the cells of the epidermis and/or of the dermis, via an effective mechanical effect at the surface of the skin (stratum corneum).

The expression “effective mechanical effect at the surface of the skin” is intended to mean the ability of a cosmetic agent to induce biologically effective mechanical tensions, i.e. mechanical tensions capable of transmitting a mechanical disturbance from cell to cell or via the extracellular matrix, and involving the activation of mechanoreceptors present on the membranes of said cells. The cells are referred to as “biologically sensitive to mechanical tensions”: interest is attached especially to the cells of the epidermis and of the dermis, and in particular to the keratinocytes and to the fibroblasts.

These mechanical tensions, in contrast to a conventional stimulation by soluble molecules as used up until now, have the effect of modifying, by means of membrane receptors or “mechanoreceptors”, an equilibrium which is established between the extracellular matrix and a cell, or between two neighbouring cells.

The mechanical tensions are transmitted in the cell in the form of biochemical signals via membrane receptors or mechanoreceptors.

These mechanoreceptors are membrane receptors sensitive to mechanical tensions, i.e. membrane receptors capable of inducing an intracellular biological response in response to a mechanical disturbance. They include integrins (Pommerenke et al., Eur J Cell Biol 1996 June; 70(2): 157-64), receptors of the PECAM1 type (Fujiwara et al., Cell struct funct 2001 February; 26(1): 11-7) or else PDGF growth factor receptors (Li et al., Cell Signal 2000 July; 12(7): 435-45).

It is in this context that the Applicant proposes to use, in combination with these tensing agents or devices, saccharide compounds which induce and/or increase the expression and therefore the number of mechanoreceptors in skin cells, in order to increase the capacity of said cells to respond to mechanical solicitations and thus to potentiate and/or increase and/or prolong the biological response induced by these tensing agents. This combination is, moreover, advantageous in that it makes it possible to limit the effective amount of tensing agents that is necessary in order to obtain the desired biological effect, thus making it possible to optimize the comfort of the cosmetic compositions containing them.

According to the invention, the term “mechanoreceptors” is in particular intended to mean membrane receptors sensitive to mechanical tensions, i.e. membrane receptors capable of inducing an intracellular biological response in response to a mechanical disturbance.

They include integrins, receptors of the PECAM1 type or else PDGF growth factor receptors.

Interest will especially be attached to the integrin group, and in particular to the class of β1 integrins involved in the sensitivity of cells to mechanical stresses.

Integrins are adhesion molecules involved in cell-cell and cell-matrix interactions. They are heterodimeric receptors composed of two subunits α and β associated non-covalently. More than 17 chains of the α-subunit and 8 chains of the β-subunit have been described, which chains associate to form 23 different heterodimers.

The transmembrane domain of the α-subunits consists of an α-helix, which is very conserved from one subunit to the other, responsible for the function of anchoring the integrin to the membrane, and participates in signal transduction.

The cytoplasmic domain of the β-subunits, which is very conserved from one subunit to the other, is responsible, firstly, for the formation of the heterodimer and, secondly, for the binding with structural proteins of the cytoskeleton; this association also regulates signal transduction.

The integrin heterodimers can be categorized according to their substrate; it is in particular known that:
α1β1 and α2β1 heterodimers bind to collagen;
α4β1, α5β1, α8β1 and αvβ1 heterodimers bind to fibronectin;
α1β1, α2β1, α3β1 and α6⊕1 heterodimers bind to laminins.

Collagen, fibronectin and laminins are matrix proteins or proteins of the extracellular matrix, which participate in cell adhesion and play an important role in cell migration and cell signalling. During the processes of cell adhesion and cell migration, the cells interact with the matrix molecules via membrane receptors, and in particular integrins such as those described above. This interaction initiates intracellular responses involved in cell signalling, cell differentiation, cell migration and/or cell proliferation.

The present invention therefore relates in particular to a composition comprising, in a physiologically acceptable medium, at least one saccharide compound which increases the expression of mechanoreceptors in skin cells, and at least one tensing agent, said saccharide compound being different from the tensing agent.

According to an alternative, it relates to a kit comprising (a) at least one composition comprising a saccharide compound which increases the expression of mechanoreceptors in skin cells and (b) at least one tensing device.

### Tensing Agent

The term “tensing agent” that can be used according to the invention is intended to mean compounds capable of having a tensing effect on the skin, i.e. which can tighten the skin.

In general and according to this first embodiment, the term “tensing agent” according to the invention is intended to mean all compounds which are soluble or dispersible in water at a temperature ranging from 25° C. to 50° C. at a concentration of 7% by weight in water or at the maximum concentration at which they form a medium with a homogeneous appearance, and which produce, at this concentration of 7% or at this maximum concentration in water, a retraction of more than 15% in the test described hereinafter.

The maximum concentration at which they form a medium with a homogeneous appearance is determined to within ±10%, and preferably to within ±5%.

The term “medium with a homogeneous appearance” is intended to mean a medium which does not show any aggregates visible to the naked eye.

For the determination of said maximum concentration, the tensing agent is gradually added to water with stirring using a deflocculating mixer at a temperature ranging from 25° C. to 50° C., and the mixture then continues to be stirred for one hour. Then, after 24 hours, the mixture thus prepared is observed in order to determine whether it has a homogeneous appearance (absence of aggregates visible to the naked eye).

The tensing effect can be characterized by means of an in vitro retraction test.

A homogeneous mixture of the tensing agent in water, at a concentration of 7% by weight or at the maximum concentration defined above, is prepared beforehand and as described above.

30 μl of the homogeneous mixture is deposited on a rectangular test specimen (10×40 mm, therefore having an initial width W₀ of 10 mm) of an elastomer having an elasticity modulus of 20 MPa and a thickness of 100 μm.

After drying for 3 h at 22±3° C. and 40±10% relative humidity RH, the test specimen of elastomer shows a retracted width, noted W₃ₕ, due to the tension exerted by the tensing agent deposited.

The tensing effect (TE) of said agent is then quantified in the following way:

‘TE’=(W₀−W₃ₕ/W₀)×100 in %
with W₀=initial width 10 mm
and W₃ₕ=width after drying for 3 h TE=(WO-W₃h/WO)xlOO in % with W₀=initial width 10 mm and W₃h=width after drying for 3 h

The tensing agent can be chosen from:

a) plant or animal proteins and hydrolysates thereof;
b) polysaccharides of natural origin;
c) mixed silicates;
d) colloidal particles of inorganic fillers;
e) synthetic polymers;
and mixtures thereof.

Those skilled in the art will be able to choose, from the chemical categories listed above, the materials which correspond to the tensing test as described above.

These various categories of tensing agents will now be described.

a) Plant Proteins and Hydrolysates Thereof.

Examples of plant proteins and plant protein hydrolysates that can be used as tensing agents according to the invention consist of maize, rye, Triticum, aestivum, buckwheat, sesame, spelt, tobacco, pea, bean, lentil, soybean, almond and lupin proteins and protein hydrolysates.

As animal proteins that can be used according to the invention, mention may in particular be made of proteins extracted from silk, from milk, from whey and from egg.

b) Polysaccharides

The polysaccharides that are suitable for the formulation of the compositions according to the invention are any polysaccharides of natural or synthetic origin capable of forming gels either of thermoreversible type or of crosslinked type. These polysaccharides have a high molecular weight, generally ranging from 100 to 10 000 kD.

Polysaccharides capable of forming thermoreversible gels will preferably be used. The term “thermoreversible” is intended to mean the fact that the gel state of these polymer solutions is obtained reversibly once the solution has cooled to below the gelling temperature characteristic of the polysaccharide used.

A first family of polysaccharides of natural origin that can be used in the present invention consists of carrageenans, and most particularly kappa-carrageenan and iota-carrageenan. They are linear polysaccharides present in certain red algae. They consist of alternating β-1,3 and α-1,4 galactose residues, it being possible for numerous galactose residues to be sulphated. This family of polysaccharides is described in chapter 3 of the book “Food Gels” edited by Peter HARRIS, Elsevier 1989.

Another family of polysaccharides that can be used consists of agars. They are also polymers extracted from red algae and they consist of alternating 1,4-L-galactose and 1,3-D-galactose residues. This family of polysaccharides is also described in chapter 1 of the book “Food Gels” mentioned above.

A third family of polysaccharides consists of polysaccharides of bacterial origin, called gellans. They are polysaccharides consisting of alternating glucose, glucuronic acid and rhamnose residues. These gellans are described in particular in chapter 6 of the book “Food Gels” mentioned above. Finally, in the case of polysaccharides which form gels of crosslinked type, in particular induced by the addition of salts, mention will be made of polysaccharides belonging to the alginate and pectin family.

These tensing polysaccharides may or may not be present in the form of microgels as described in application FR 2 829 025.

Mention may also be made of tensing systems comprising:
a polysaccharide and a polyhydroxylated moisturizing agent, such as the system described in application FR 2 828 810;
a polysaccharide of pullulan type, as described in patent U.S. Pat. No. 6,703,027;
a polysaccharide of Biopolymer B16 type, as described in patent U.S. Pat. No. 5,175,279.

c) Mixed Silicates

Another class of tensing agents that can be used according to the invention consists of mixed silicates. This expression is intended to mean any silicates of natural or synthetic origin which contain at least two different cations chosen from alkali metals (for example, Na, Li or K) or alkaline earth metals (for example, Be, Mg or Ca) and transition metals.

Phyllosilicates, i.e. silicates which have a structure in which the SiO₄ tetrahedrons are organized in sheets between which the metal cations are trapped, are preferably used.

A family of silicates that is particularly preferred as tensing agents is the laponite family. Laponites are magnesium, lithium and sodium silicates which have a layered structure similar to that of montmorillonites. Laponite is the synthetic form of the natural mineral called “hectorite”. The laponite sold under the name Laponite XLS or Laponite XLG by the company ROCKWOOD can, for example, be used.

In the specific case of laponites, a concentration much lower than 7% will be used in the retraction test described above.

d) Colloidal Particles of Inorganic Filler

The term “colloidal particles” is intended to mean particles in the form of a dispersion in an aqueous, aqueous-alcoholic or alcoholic, preferably aqueous, medium, and having a number-average diameter of between 0.1 and 100 nm, preferably between 3 and 30 nm.

The colloidal particles according to the invention have no thickening property in water, alcohol, oil or any other solvents. At a concentration of greater than or equal to 15% by weight in water, the viscosity of the solutions thus obtained is less than 0.05 Pa·s for a shear rate equal to 10 s⁻¹. The measurements are carried out at 25° C. using a Haake RheoStress RS150 rheometer in the cone-plate configuration, the measurements of the measuring cone being: diameter: 60 mm and angle: 2°.

These particles are generally prepared according to a sol-gel process and therefore differ in particular from particles of fumed silica, which agglomerate in water so as to form aggregates larger in size.

Colloidal particles of inorganic filler that can be used according to the invention are generally chosen from colloidal particles of silica, of cerium oxide, of zirconium oxide, of alumina, of calcium carbonate, of barium sulphate, of calcium sulphate, of zinc oxide and of titanium dioxide, colloidal particles of platinum and mixed colloidal particles, for instance titanium dioxides coated one or more times, such as silica-coated titanium dioxide. Colloidal silicas or silica-alumina composite colloidal particles will preferably be used in the composition according to the invention.

### Colloidal Particles of Silica

For the purpose of the application, the term “colloidal silicas” is intended to mean colloidal particles of silica in the form of a dispersion in an aqueous, aqueous-alcoholic or alcoholic medium. The colloidal particles of silica have a diameter ranging from 0.1 to 100 nm, and preferably from 3 to 30 nm. These particles are in the form of aqueous dispersions and have no thickening property in water, alcohol, oil or any other solvents. At a concentration of greater than or equal to 15% by weight in water, the viscosity of the solutions thus obtained is less than 0.05 Pa·s for a shear rate equal to 10 s⁻¹. The measurements are carried out at 25° C. using a Haake RheoStress RS150 rheometer in the cone-plate configuration, the measurements of the measuring cone being: diameter: 60 mm and angle: 2°.

As colloidal silicas that can be used in the composition according to the invention, mention may, for example, be made of those sold by the company Catalysts and Chemicals under the names Cosmo S-40 and Cosmo S-50.

### Silica-Alumina Composite Colloidal Articles

The colloidal particles of inorganic fillers that can be used according to the invention can also be chosen from silica-alumina composite colloidal particles. The term “silica-alumina composite” is intended to mean particles of silica in which aluminium atoms have been substituted in part with silica atoms. For the purpose of the application, the term “colloidal particles” is intended to mean colloidal particles in the form of a dispersion in an aqueous, aqueous-alcoholic or alcoholic medium. The silica-alumina composite colloidal particles have a diameter ranging from 0.1 to 100 nm, and preferably from 3 to 30 nm. These particles are in the form of aqueous dispersions and have no thickening property in water, alcohol, oil or any other solvents. At a concentration of greater than or equal to 15% by weight in water, the viscosity of the solutions thus obtained is less than 0.05 Pa·s for a shear rate equal to 10 s⁻¹. The measurements are carried out at 25° C. using a Haake RheoStress RS150 rheometer in the cone-plate configuration, the measurements of the measuring cone being: diameter: 60 mm and angle: 2°.

At a pH of 7, the silica-alumina composite colloidal particles according to the invention have a zeta potential of less than −20 mV, and preferably less than −25 mV. The measurements are carried out at 25° C. using a DELSA 440SX device from Coulter Scientific Instrument.

As silica-alumina composite colloidal particles that can be used in the compositions according to the invention, mention may, for example, be made of those sold by the company Grace under the names Ludox AM, Ludox AM-X 6021, Ludox HSA and Ludox TMA.

e) Synthetic Polymers

The synthetic polymers used according to the invention can be in solution or in suspension in a polar liquid or apolar liquid (latex), in particular in an aqueous solution or aqueous dispersion, or in a dry form that can be redispersed in a cosmetic solvent.

The synthetic polymers that can be used as a tensing agent can be chosen from:
polycondensates, in particular polyurethanes;
acrylic polymers;
graft silicone polymers;
water-soluble or water-dispersible polymers comprising water-soluble or water-dispersible units and units with an LCST.

The synthetic polymers according to the invention can in particular be chosen from interpenetrating polymer networks (IPNs).

These polymers can in particular be in the form of random linear copolymers, interpenetrating polymer networks (IPNs), polycondensates, graft silicone polymers and block polymers. Regardless of its nature, the synthetic polymeric tensing agent can have a weight-average mass Mw ranging from 3000 to 1 000 000 Da.

### Random Linear Copolymers

For the purpose of the present invention, the tensing random linear copolymers are chosen from random copolymers comprising a linear main chain of ethylenic nature having a molecular weight of less than 600 000 Da (g/mol), preferably a molecular weight, by weight, of between 15 000 and 600 000 g/mol, and contain at least 70% of a monomer with a glass transition temperature Tg of greater than 40° C. (preferably >60° C.), the corresponding homopolymer of which is insoluble in water at 25° C., and at least one ionic hydrophilic monomer. This copolymer can also contain a monomer, which is not predominant, with a Tg of less than 40° C.

These polymers generally have an overall glass transition temperature of greater than or equal to 45° C.

The preferred copolymers are all those which consist:
of 70% to 90% by weight overall of at least one alkyl or aryl acrylate and/or at least one alkyl or aryl methacrylate mentioned in the list below and/or of styrene;
of 10% to 30% by weight of (meth)acrylic acid.

List of preferred alkyl(meth)acrylates: benzyl acrylate, cyclohexyl acrylate, tert-butyl acrylate, isobornyl acrylate and norbornyl acrylate, methyl methacrylate, ethyl methacrylate, isobutyl methacrylate, cyclohexyl methacrylate, benzyl methacrylate, tert-butyl methacrylate, isobornyl methacrylate and norbornyl methacrylate, preferably methyl methacrylate and cyclohexyl methacrylate.

Among the polymers mentioned above, preference will particularly be given to:
methyl methacrylate/methacrylic acid copolymers; methyl methacrylate/acrylic acid copolymers, said copolymers containing between 70% and 90% by weight of methyl methacrylate;
ethyl methacrylate/methacrylic acid copolymers; ethyl methacrylate/acrylic acid copolymers, said copolymers containing between 70% and 90% by weight of ethyl methacrylate;
isobutyl methacrylate/methacrylic acid copolymers; isobutyl methacrylate/acrylic acid copolymers, said copolymers containing between 70% and 90% by weight of isobutyl methacrylate;
benzyl methacrylate/methacrylic acid copolymers; benzyl methacrylate/acrylic acid copolymers, said copolymers containing between 70% and 90% by weight of benzyl methacrylate;
benzyl acrylate/methacrylic acid copolymers; benzyl acrylate/acrylic acid copolymers, said copolymers containing between 70% and 90% by weight of benzyl acrylate;
cyclohexyl methacrylate/methacrylic acid copolymers; cyclohexyl methacrylate/acrylic acid copolymers, said copolymers containing between 70% and 90% by weight of cyclohexyl methacrylate;
cyclohexyl acrylate/methacrylic acid copolymers; cyclohexyl acrylate/acrylic acid copolymers, said copolymers containing between 70% and 90% by weight of cyclohexyl acrylate;
tert-butyl methacrylate/methacrylic acid copolymers; tert-butyl methacrylate/acrylic acid copolymers, said copolymers containing between 70% and 90% by weight of tert-butyl methacrylate;
tert-butyl acrylate/methacrylic acid copolymers; tert-butyl acrylate/acrylic acid copolymers, said copolymers containing between 70% and 90% by weight of tert-butyl acrylate;
isobornyl methacrylate/methacrylic acid copolymers; isobornyl methacrylate/acrylic acid copolymers, said copolymers containing between 70% and 90% by weight of isobornyl methacrylate;
isobornyl acrylate/methacrylic acid copolymers; isobornyl acrylate/acrylic acid copolymers, said copolymers containing between 70% and 90% by weight of isobornyl acrylate;
norbornyl methacrylate/methacrylic acid copolymers; norbornyl methacrylate/acrylic acid copolymers, said copolymers containing between 70% and 90% by weight of norbornyl methacrylate;
norbornyl acrylate/methacrylic acid copolymers; norbornyl acrylate/acrylic acid copolymers, said copolymers containing between 70% and 90% by weight of norbornyl acrylate; and
styrene/methacrylic acid copolymers; styrene/acrylic acid copolymers, said copolymers containing between 70% and 90% by weight of styrene.

The copolymers in accordance with the present invention are in the form of a dispersion in a polar liquid. These copolymers are dispersed in water after neutralization with a base.

A preferred copolymer according to the invention is chosen from methyl methacrylate/methacrylic acid copolymers, said copolymers containing between 70% and 90% by weight of methyl methacrylate.

### Interpenetrating Polymers

For the purpose of the present invention, the term “interpenetrating polymer network” is intended to mean a blend of two intermeshed polymers, obtained by simultaneous polymerization and/or crosslinking of two types of monomer, the blend obtained having a single glass transition temperature. Examples of IPNs that are suitable for use in the present invention, and also the process for preparing them, are described in patents U.S. Pat. No. 6,139,322 and U.S. Pat. No. 6,465,001, for example. Preferably, the IPN according to the invention comprises at least one acrylic polymer, and it more preferably also comprises at least one polyurethane or a copolymer of vinylidene fluoride and of hexafluoropropylene. According to a preferred embodiment, the IPN according to the invention comprises a polyurethane polymer and a polyacrylic polymer. Such IPNs are in particular those of the Hybridur series that are commercially available from the company Air Products. An IPN that is particularly preferred is in the form of an aqueous dispersion of particles with a weight-average size of between 90 and 110 nm, and a number-average size of approximately 80 nm. This IPN preferably has a glass transition temperature, Tg, that ranges from approximately −60° C. to +100° C. An IPN of this type is in particular sold by the company AIR PRODUCTS under the trade name Hybridur 875. Other IPNs that are suitable for use in the present invention are referenced Hybridur X01602 and Hybridur 580.

Other IPNs that are suitable for use in the present invention include IPNs consisting of a blend of a polyurethane with a copolymer of vinylidene fluoride and of hexafluoropropylene. These IPNs can in particular be prepared as described in patent U.S. Pat. No. 5,349,003. As a variant, they are commercially available in the form of a colloidal dispersion in water, in a ratio of the fluoro copolymer to the acrylic polymer of between 70:30 and 75:25, under the trade names Kynar RC-10,147 and Kynar RC-10,151 from the company Atofina.

### Polycondensate

The composition can, according to a second variant, comprise at least one polycondensate as synthetic polymeric tensing agent. Polymers in the form of polycondensates which have a tensing effect are in particular described in application WO 98/29092.

As polycondensates, mention may be made of polyurethanes, in particular anionic, cationic, non-ionic or amphoteric polyurethanes, polyurethane acrylics, polyurethane polyvinylpyrrolidones, polyester polyurethanes, polyether polyurethanes, polyureas, and blends thereof.

The polyurethane may, for example, be a polyurethane, polyurea/urethane or polyurea copolymer which is aliphatic, cycloaliphatic or aromatic, which comprises, alone or as a mixture,
at least one block originating from a linear or branched aliphatic and/or cycloaliphatic and/or aromatic polyester, and/or
at least one block originating from an aliphatic and/or cycloaliphatic and/or aromatic polyether, and/or
at least one block containing fluoro groups.

The polyurethanes can also be obtained from branched or unbranched polyesters or from alkyds containing mobile hydrogens that are modified by reaction with a diisocyanate and a bifunctional organic compound (for example, a dihydro, diamino or hydroxyamino compound), further containing either a carboxylic acid or carboxylate group, or a sulphonic acid or sulphonate group, or else a neutralizable tertiary amine group or a quaternary ammonium group. Mention may also be made of polyesters, polyester amides, fatty-chain polyesters, polyamides and epoxy ester resins.

In order to form a polyurethane, mention may be made, as a monomer which carries an anionic group that can be used in polycondensation, of dimethylolpropionic acid, trimellitic acid or derivative thereof such as trimellitic anhydride, the sodium salt of the acid 3-sulphopentanediol, or the sodium salt of 5-sulpho-1,3-benzenedicarboxylic acid.

Among the polycondensates, mention may be made of the polymers sold under the trade names Avalure UR410, Avalure UR405 and Avalure UR460 by the company Noveon, and under the trade names Neorez R974, Neorez R981 and Neorez R970 by the company Avecia.

Mention may also be made of combinations of polymers, such as polyurethanes having a degree of shrinkage of less than or equal to 20% and acrylic polymers having a degree of shrinkage of less than or equal to 20%, which are described in patent application WO 2005067884.

### Graft Silicone Polymer

Among the synthetic polymeric tensing agents used in the composition according to the invention, mention may be made, as a variant, of graft silicone polymers in particular, as defined in patent application EP-1038519. The polymer in question may more particularly be a polymer comprising a main chain of silicone or polysiloxane (Si—O— polymer) on which is grafted, within said chain and, optionally, at least one of its ends, at least one organic group not containing silicone.

The polymers having a polysiloxane backbone grafted with non-silicone organic monomers according to the invention may be existing commercial products or may be obtained according to any means known to those skilled in the art, in particular by reaction between (i) a starting silicone correctly functionalized on one or more of its silicon atoms and (ii) a non-silicone organic compound which is itself correctly functionalized with a function which is capable of reacting with the functional group(s) carried by said silicone, thereby forming a covalent bond; a classic example of such a reaction is the hydrosilylation reaction between ≡Si—H groups and vinyl groups CH₂═CH—, or else the reaction between thiofunctional groups —SH and these same vinyl groups.

Examples of polymers having a polysiloxane backbone grafted with non-silicone organic monomers that are suitable for an implementation of the present invention, and also their specific method of preparation, are in particular described in patent applications EP-A-0582152, WO 93/23009 and WO 95/03776, the teachings of which are included in their entirety in the present description by way of non-limiting references.

According to a particularly preferred embodiment of the present invention, the silicone polymer that is used, having a polysiloxane backbone grafted with non-silicone organic monomers, is the result of free-radical copolymerization between, firstly, at least one non-silicone anionic organic monomer containing an ethylenic unsaturation and/or a non-silicone hydrophobic organic monomer containing an ethylenic unsaturation and, secondly, a silicone having in its chain at least one functional group capable of reacting with said ethylenic unsaturations of said non-silicone monomers, thereby forming a covalent bond, and in particular thiofunctional groups.

According to the present invention, said ethylenically unsaturated anionic monomers are preferably selected, alone or as mixtures, from linear or branched unsaturated carboxylic acids which optionally are partially or totally neutralized in the form of a salt, it being possible for this or these unsaturated carboxylic acid(s) to be, more particularly, acrylic acid, methacrylic acid, maleic acid, maleic anhydride, itaconic acid, fumaric acid and crotonic acid. The salts that are suitable are, in particular, alkali metal salts, alkaline-earth metal salts and ammonium salts. It will be noted that, likewise, in the final graft silicone polymer, the anionic organic group which constitutes the result of the free-radical (homo)polymerization of at least one anionic monomer of unsaturated carboxylic acid type may, after reaction, be post-neutralized with a base (sodium hydroxide, aqueous ammonia, etc.) in order to bring it into the form of a salt.

According to the present invention, the ethylenically unsaturated hydrophobic monomers are preferably chosen, alone or as mixtures, from esters of acrylic acid with alkanols and/or esters of methacrylic acid with alkanols. The alkanols are preferably C₁-C₁₈ and more particularly C₁-C₁₂. The preferred monomers are chosen from the group consisting of isooctyl(meth)acrylate, isononyl(meth)acrylate, 2-ethylhexyl(meth)acrylate, lauryl (meth)acrylate, isopentyl(meth)acrylate, n-butyl(meth)acrylate, isobutyl(meth)acrylate, methyl(meth)acrylate, tert-butyl(meth)acrylate, tridecyl(meth)acrylate, stearyl (meth)acrylate and mixtures thereof.

One class of silicone polymers having a polysiloxane backbone grafted with non-silicone organic monomers that is particularly suitable for the implementation of the present invention consists of silicone polymers comprising in their structure the unit of formula (I) below:

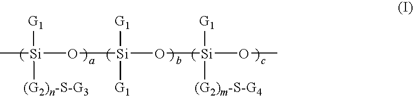

in which the radicals G₁, which may be identical or different, represent hydrogen or a C₁-C₁₀ alkyl radical or else a phenyl radical; the radicals G₂, which may be identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer; m and n are, independently of one another, equal to 0 or 1; a is an integer ranging from 0 to 50; b is an integer that may be between 10 and 350; c is an integer ranging from 0 to 50; with the proviso that one of the parameters a and c is other than 0.

Preferably, the unit of formula (I) above has at least one, and even more preferably all, of the following features:
the radicals G₁ denote a C₁-C₁₀ alkyl radical;
n is non-zero, and the radicals G₂ represent a divalent C₁-C₃ radical;
G₃ represents a polymeric radical resulting from the (homo)polymerization of at least one ethylenically unsaturated carboxylic acid monomer, preferably acrylic acid and/or methacrylic acid;
G₄ represents a polymeric radical resulting from the (homo)polymerization of at least one C₁-C₁₀ alkyl(meth)acrylate monomer.

Examples of graft silicone polymers corresponding to formula (I) are thus in particular polydimethylsiloxanes (PDMSs) grafted, via a thiopropylene linker, with polymer units of poly(meth)acrylic acid type and/or of poly-alkyl, in particular C₁-C₃, or even C₁, alkyl, (meth)acrylate type.

These polymers are referenced under the CTFA name “polysilicone-8”.

A preferred example of a graft silicone polymer is the polysilicone-8 (CTFA name) which is a polydimethylsiloxane grafted, via a thiopropylene linker, with mixed polymer units of poly(meth)acrylic acid type and of poly-alkyl, in particular C₁-C₃, or even C₁, alkyl, (meth)acrylate type.

The polymer in question may thus be a propylthio(polymethyl acrylate/methyl methacrylate/methacrylic acid)-grafted polydimethylsiloxane or a propylthio(polymethyl acrylate)-, propylthio(polymethyl methacrylate)- and propylthio(polymethacrylic acid)-grafted polydimethylsiloxane. As a variant it may be a propylthio(polyisobutyl methacrylate)- and propylthio(polymethacrylic acid)-grafted polydimethylsiloxane. Use is preferably made of a propylthio(polymethyl acrylate/methyl methacrylate/methacrylic acid)-grafted polydimethylsiloxane.

A polymer of this type is in particular available under the trade name VS 80 or VS 70 (at 10% in water) or LO 21 (in pulverulent form) from the company 3M.

Preferably, the number-average molecular mass of the silicone polymers having a polysiloxane backbone grafted with non-silicone organic monomers of the invention ranges from 10 000 to 1 000 000 approximately, and even more preferably from 10 000 to 100 000 approximately.

### Star Polymer

According to yet another possibility, the synthetic polymeric tensing agent that can be used in the composition according to the invention can comprise at least one polymer of “star” structure, represented by the following formula (I):

A−[(M1)p1−(M2)p2 . . . (Mi)pi]n (I)

in which:
A represents a multifunctional centre, of functionality “n”, n being an integer greater than 2, in particular greater than 5;
[(M1)p1−(M2)p2 . . . (Mi)pj] represents a polymeric chain, also called a “branch”, consisting of polymerized monomers Mi, which may be identical or different, having a polymerization index pj, each branch being identical or different, and being grafted covalently onto said centre A,
i is greater than or equal to 1, and pj is greater than or equal to 2;
   said polymer comprising one or more monomers Mi whose corresponding homopolymer has a Tg of greater than or equal to approximately 10° C., preferably greater than or equal to 15° C., and even better still greater than or equal to 20° C.; this or these monomer(s) Mi being present in a minimum amount of approximately 45% by weight, preferably in an amount ranging between 55% and 99% by weight, and even better still between 75% and 90% by weight, relative to the total weight of the entirety of the monomers of the final polymer. These polymers, and the process for preparing them, are described in particular in document EP 1 043 345.

### Block Polymer

As a variant, the synthetic polymeric tensing agents that can be used in the composition according to the invention can be polystyrene (PS)-polyethyl acrylate (PEA) block polymers.

Very generally, the term “block polymer” is intended to mean a polymer consisting of at least two distinct homopolymers consisting solely of monomers A and B respectively. Thus, the blocks according to the invention are, respectively, polystyrene (PS) and polyethyl acrylate (PEA) blocks.

In the context of this variant, the polymer may be a triblock polymer of PS-PEA-PS type or else a multiblock polymer of PS-[PEA-PS]n, or PEA-[PS-PEA]n type, where n is a positive integer, and preferably is equal to 1. Advantageously, these block polymers are linear copolymers. The molecular weight of this polymer is preferably greater than 10 000 Daltons, and even more preferably greater than 50 000 Daltons. The weight ratio of the PS and PEA monomers may be defined such that PS/PEA is greater than 1, and preferably such that PS/PEA is greater than 5.

Mention may be made of the triblock polymer PS(30 000)-PEA(10 000)-PS(30 000), which is most particularly suitable for the implementation of the invention. This particularly advantageous block copolymer is a triblock copolymer comprising:
a first block comprising units deriving from styrene, having a number-average molecular mass of 30 000 g/mol;
a second block consisting of units deriving from ethyl acrylate, having a number-average molecular mass of 10 000 g/mol;
a third block comprising units deriving from styrene, having a number-average molecular mass of 30 000 g/mol.

A copolymer corresponding to the definition given above may be a copolymer for which the first block and/or the third block, and preferably the first block and the third block comprise, in addition to the units deriving from styrene, units deriving from methacrylic acid, for example, in a mass ratio (styrene/methacrylic acid) of 98/2.

The synthetic copolymers used according to the invention may also, as a variant, consist of a random polystyrene/polyethyl acrylate copolymer. The weight ratio of the PS and PEA monomers is itself defined such that PS/PEA>1, and preferably such that PS/PEA>5.

Alternatively, the tensing polymers according to the invention can also be selected from vinyl derivatives such as polyvinyl alcohols and polyvinylpyrrolidones, in either block or else in random form.

Finally, synthetic polymers which are appropriate may be water-soluble or water-dispersible polymers containing water-soluble or water-dispersible units and containing LCST units, said LCST units exhibiting, in particular, a separation temperature in water of 5 to 40° C. and a mass concentration of 1%. This type of polymer is more fully described in patent application FR 2 819 429.

According to a particularly preferred embodiment of the invention, the tensing agent is chosen from:
interpenetrating polymers, in particular comprising a polyurethane polymer and a polyacrylic polymer, such as those sold under the name Hybridur, and in particular Hybridur 875; and
synthetic polymers, in particular acrylic polymers of latex type, such as those described in the examples hereinafter.

According to a specific embodiment of the invention, the tensing agent used according to the invention may be chosen from:
plant or animal proteins and hydrolysates thereof;
polysaccharides of natural origin;
synthetic polymers of polycondensate or graft silicone polymer type,
and mixtures thereof.

Preferably, use will be made of a tensing agent chosen from silicone polymers comprising a polysiloxane backbone grafted with non-silicone organic monomers.

In particular, the silicone polymer comprises in its structure the unit of formula (I) below:

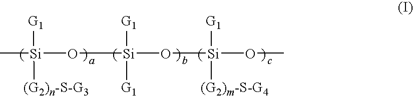

in which the radicals G₁, which may be identical or different, represent hydrogen or a C₁-C₁₀ alkyl radical or else a phenyl radical; the radicals G₂, which may be identical or different, represent a C₁-C₁₀ alkylene group; G₃ represents a polymeric residue resulting from the (homo)polymerization of at least one ethylenically unsaturated anionic monomer; G₄ represents a polymeric residue resulting from the (homo)polymerization of at least one ethylenically unsaturated hydrophobic monomer; m and n are, independently of one another, equal to 0 or 1; a is an integer ranging from 0 to 50; b is an integer that may be between 10 and 350; c is an integer ranging from 0 to 50; with the proviso that one of the parameters a and c is other than 0.

Preferably, the unit of formula (I) has at least one, and even more preferably all, of the following features:
the radicals G₁ denote a C₁-C₁₀ alkyl radical;
n is non-zero, and the radicals G₂ represent a divalent C₁-C₃ radical;
G₃ represents a polymeric radical resulting from the (homo)polymerization of at least one ethylenically unsaturated carboxylic acid monomer, preferably acrylic acid and/or methacrylic acid;
G₄ represents a polymeric radical resulting from the (homo)polymerization of at least one C₁-C₁₀ alkyl(meth)acrylate monomer.

Also in particular, the graft silicone polymers corresponding to formula (I) are polydimethylsiloxanes (PDMSs) grafted, via a thiopropylene linker, with polymer units of the poly(meth)acrylic acid type and/or alkyl, in particular C₁-C₃, or even C₁, alkyl, poly(meth)acrylate type.

A preferred graft silicone polymer is a propylthio(polymethyl acrylate/methyl methacrylate/methacrylic acid)-grafted polydimethyl siloxane.

According to another preferred embodiment of the invention, use will be made of a tensing agent chosen from:
(i) mineral tensing agents, such as mixed silicates and colloidal particles of inorganic fillers,
(ii) tensing polymers, in particular:
   methyl methacrylate/methacrylic acid copolymers, said copolymers containing between 70% and 90% by weight of methyl methacrylate;
   interpenetrating polymers comprising a polyurethane and an acrylic polymer,
and mixtures thereof.

The tensing agents which come under this preferred embodiment of the invention have the particularity of forming, when they are deposited on a glass plate, a mosaic deposit. In the remainder of the description, reference will be made to “mosaic-effect tensing agents”.

According to the invention, the term “mosaic-effect tensing agent” is intended to mean an agent which, when it is applied to a glass plate, dries to form a tessellated deposit, it being possible for the size and shape of its constituent tessellae to depend on their location relative to the edges of the deposit.

The term “tessellated deposit” is intended to mean more specifically a discontinuous deposit made up of a multitude of small individualized domains or microdomains.

The tessellae or microdomains are generally small in size. This size may range from 0.1 mm² to several mm².

In particular, such a mosaic deposit cannot be peeled or detached from the substrate, in contrast to continuous or semi-continuous deposits which adhere to the substrate, and which can be detached or peeled either in a single piece or in two or more relatively large-sized strips.

Moreover, such a mosaic deposit generally exhibits low resistance to water, i.e., on contact with water, the deposit breaks up.

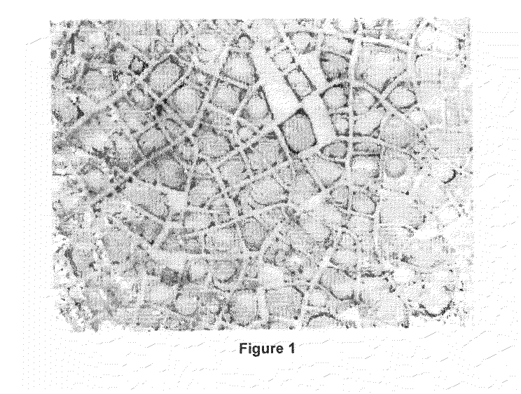

Such a mosaic deposit according to the invention is represented in FIG. 1.

The tessellated or mosaic appearance of the deposit results in particular from the fact that the stresses developed by these tensing agents in the course of drying are greater than the forces of cohesion (rigidity) of the deposit.

From a mechanical point of view, such deposits formed by these mosaic-effect tensing agents can be characterized by their property of fracturing under stresses, evaluated for example in the mechanical strength test described hereinafter.

In particular, the mosaic-effect tensing agents according to the invention form deposits characterized advantageously by a breaking energy of between 0 and 20 J/m² (preferably equal to 0) and a breaking deformation of between 0 and 0.2 mm in the mechanical strength test described below.

Said mechanical strength test consists, for example, in applying compressive stress to breaking point to the tensing agent at the surface of a flexible and deformable foam.

The tensing agent is deposited at a concentration of 7% by weight in water or at the maximum concentration by weight at which it forms, in water at a temperature ranging from 25° C. to 50° C., a medium with a homogeneous appearance.

The use of this foam substrate makes it possible to apply a substantial deformation to the surface-deposited tensing agent, and hence makes it possible to quantify its breaking strength. The mechanical compressive stress is exerted by means of a cylindrical punch 1 mm in diameter; the rate of displacement of the punch being 0.1 mm/s.

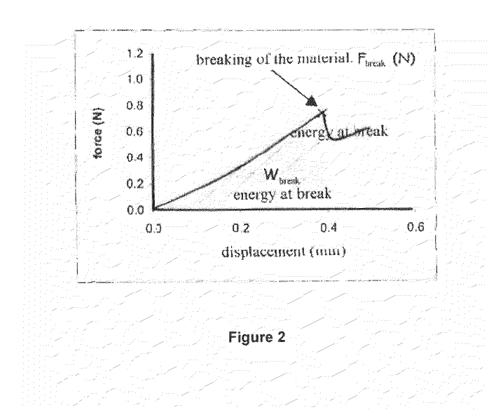

The test is carried out using a TA-XT2i texture analyser sold by the company Stable Micro System. A curve of force F (in N) as a function of the displacement d (in mm) is thus obtained, from which it is possible to determine the breaking point of the material (tensing agent) or breaking energy W_{break}, expressed in J/m², as represented in FIG. 2.

The breaking energy W_{break} expressed in J/m² corresponds to the area under the curve F=f(d) obtained at the value of the displacement for which a discontinuation F_{break} (N) is observed.

The mosaic-effect tensing agents used according to the invention are advantageous relative to the other tensing agents, which form a continuous or semi-continuous deposit which adheres to a flexible substrate such as the skin, in that they allow better distribution, over the whole surface of the skin, of the tensions exerted, relative to a continuous or semi-continuous deposit.

This is because, in the case of a continuous or semi-continuous deposit which adheres to a flexible substrate, the tensions develop in the substrate solely at the periphery of the solid deposit.

In contrast, in the case of a mosaic deposit according to the invention, with the same surface area as the above deposit, the tensions develop at the periphery of each independent tessella of the deposit, thereby considerably increasing the surface area subject to the tensions.

By virtue of these tensions being more effectively distributed over the entire surface area of the skin on which the deposit is formed, the Applicant has been able to show that the biological effects are obtained much more rapidly: starting from the first hours after application of said mosaic-effect tensing agent to the skin, and in particular after 48 hours.

Preferably, colloidal particles of silica will be used as mosaic-effect tensing agent.

As other mosaic-effect tensing agents that are preferred, mention may be made of:
methyl methacrylate/methacrylic acid copolymers, said copolymers containing between 70% and 90% by weight of methyl methacrylate;
interpenetrating polymers comprising a polyurethane and an acrylic polymer.

The tensing agents preferred for use are interpenetrating polymers comprising a polyurethane and an acrylic polymer.

The tensing agent will be present in the composition in an amount that is effective for obtaining the desired biological effect according to the invention. This effective amount will be defined such that the combination of the tensing agent with the saccharide compound which increases the expression of mechanoreceptors in cells makes it possible to obtain the desired biological effect, i.e., in particular, an effect on skin homeostasis.

This effective amount or effective dose can, for example, be evaluated according to a DNA array method as described in the illustrative examples hereinafter, the general principle of which is the following:
various doses of tensing agent and of saccharide compound which increases the expression of mechanoreceptors are applied, in combination, to cells in culture or to a model of reconstructed epidermis and/or skin;
the mRNA is extracted from said treated or non-treated (control) cells and a “reverse” transcription is carried out using, for example, oligo dT and a P33-labelled deoxynucleotide triphosphate, in order to obtain labelled target cDNA sequences;
these target cDNA sequences are hybridized on dedicated minichips containing DNAs specific for the markers involved in the physiology of skin cells, and in particular in skin homeostasis (called “probe cDNAs”);
after washing, the amount of labelled target sequences is measured and is compared to the control in order to evaluate the variation in expression of the target genes which is induced by the topical application of said tensing agent, relative to the control;
next, the combined effective amounts or doses of tensing agents and of saccharide compound which increases the expression of mechanoreceptors, for which a variation is obtained in the expression of genes involved in the proliferation (increase) and/or the differentiation (decrease) of skin cells, relative to a control (non-treated), are selected. The combined effective doses for which a decrease is obtained in the expression of genes involved in keratinocyte differentiation (for example: corneodesmosin, loricrin, suprabasin) and/or an increase is obtained for genes involved in skin regeneration (for example: cytokeratins), relative to a control, preferably a variation in the expression by a factor of 2 or more relative to the control, are advantageously selected.

By way of example, the tensing agent may be included in the composition according to the invention at a content ranging from 0.01% to 30% by weight of active material, in particular from 1% to 30%, preferably from 1% to 20%, relative to the total weight of the composition.

In particular, use may be made of an effective amount of a tensing agent ranging from 2% to 30% by weight, in particular from 3% to 20%, preferably from 4% to 20% by weight of active material, relative to the total weight of the composition, for example an amount of between 6% and 10% by weight of active material, relative to the total weight of the composition.

According to a specific embodiment, use will be made of an effective amount of tensing agent of from 3% to 20% by weight of active material, relative to the total weight of the composition, preferably from 3% to 7% by weight of active material, relative to the total weight of the composition.

The term “active material” is intended to exclude the medium in which the tensing agent is optionally solubilized or in the form of a dispersion in its commercial form, for example in the case of dispersions of colloidal particles.

According to a second embodiment, a tensing device is used as a replacement for and/or as a supplement to the tensing agent.

### Tensing Device

According to the invention, the term “tensing device” is intended to mean a device intended to apply and/or maintain, in a controlled manner, mechanical stresses to and/or on the skin.

The expression “application and/or maintenance, in a controlled manner, of mechanical stresses” is intended to mean a stimulation of the skin using a device, it being possible for said device to be applied to skin not subjected to a mechanical stress (this will be described as application of a stress), or to skin already subjected to a mechanical stress, in particular of tension or traction type (in this case, it will be described as maintenance of the stress).

These mechanical stresses may be the direct or indirect result of the application to and/or the maintenance on the skin of a mechanical stimulation, electrical stimulation or ultrasonic stimulation, or combination thereof, using at least one device.

The term “indirect result” is intended to mean, for example, a mechanical stress induced by a stimulation of the subcutaneous muscles, for example of electrical type (for example: electrostimulation), using a device.

The term “mechanical stresses” is intended to mean in particular, according to the invention, a stress chosen from a tension, a traction and a pressure, and combinations thereof.

In particular, the device, according to the invention, intended to apply and/or maintain mechanical stresses to and/or on the skin is chosen from a device which induces a mechanical stimulation, an electrical stimulation or an ultrasonic stimulation, and combinations thereof.

Certain devices may be favoured according to the mechanical stress that they induce and the intended application.

By way of example, use will preferably be made of a device which generates tractions for the purpose of inducing/stimulating cell renewal and thus of combating impairments related to ageing.

In addition, use may preferably be made of devices which generate pressures in order to reduce the accumulation of extracellular matrix and thus promote regeneration of the skin and/or its cicatrization, in particular by combating the development of hypertrophic scars or keloids.

The stimulation of the skin according to the invention by means of a device for applying and/or maintaining mechanical stresses may be the result of a mechanical, electrical or ultrasonic action, or a combination thereof.

Preferably, use will be made of a stimulation induced by a mechanical device.

### Stimulation Induced by a Mechanical Device

According to a first embodiment, the device which induces a mechanical stimulation is a massage instrument or a pressure applicator.

### a) Massage Instruments or Pressure Applicators

These devices may be manual, i.e. they do not require, in order to operate, any outside energy supply other than a simple application of the device to the skin, optionally with a back-and-forth movement (massage), or may be provided with an electric motor that can be both mains-operated or battery-operated.

According to a first embodiment, the device may be a massage instrument chosen from a manual massage instrument or a massage instrument requiring an outside energy supply.

By way of example of manual instruments, mention will be made of the apparatus developed or sold by the companies: Environ (Environ Cosmetic Roll-Cit®); Repechage (Repechage Facial Lift On The Go®); Leaf & Rusher (Leaf & Rusher DermaRoller®).

These instruments may be in the form of massage rollers or balls optionally containing spikes at their surface.

By way of example of instruments requiring an outside energy supply, mention will be made of the apparatus developed or sold by the companies: WinHealth (Magic Youth Wand Eye Roller®); HoMedics (FAC-100 Facial Spa®); LPG (Lift6®).

These massage instruments may be assisted mechanically, i.e. may comprise manual control means, comprising a treatment head connected to a suction circuit, said treatment head comprising two mutually opposed surfaces, and said suction circuit allowing the formation, as a result of the reduced pressure created, of a fold of skin which will then be displaced over the surface of the human body.

Apparatus such as the Lift6® make it possible to carry out specific massages, which are obtained using massage heads connected to a suction circuit. The massage heads comprise a casing, a chamber and two transverse surfaces consisting of rollers driven positively in rotation; such apparatus is described, for example, in French patent application Nos. FR2579100, FR2589726, FR2612395, FR2723310, FR2752159, FR2768051 and FR2809952.

As an example of a mechanical stimulation device which generates a pressure, mention may be made of an apparatus comprising an applicator that is configured to apply a pressure pulse to the surface of the skin, having at least one phase of negative pressure relative to the ambient pressure, such as that described in application WO 0697925.

This phase of negative pressure relative to the ambient pressure may have a duration of 0.1 to 100 milliseconds and an intensity of 0.1 bar to 10 bar below ambient pressure.

### b) Tension-Maintaining Supports, in Particular Adhesive Supports

According to another embodiment, the device intended to generate and/or maintain mechanical stresses on the skin, in particular to maintain tensions already applied to the skin, is a support, in particular an adhesive support, capable of maintaining a tension on the skin.

This support will be applied to a stretched or drawn area of skin so as to maintain the skin under tension.

This support will in particular be characterized by an elastic modulus of greater than or equal to 500 MPa, in particular ranging from 500 to 10 000 MPa, preferably from 500 to 2000 MPa, even more preferably from 500 to 1500 MPa. This modulus may in particular be determined by dynamic mechanical analysis using, for example, the DMA 2980 (Dynamic Mechanism Analyser) sold by the company TA Instruments.

This support may, for example, be a synthetic support.

As an example of a support, in particular an adhesive support, mention may be made of patches.

Patches generally have a composite structure in the form of layers.

Advantageously, they contain a reservoir comprising the composition or the active agent which is intended to be released on the skin at the time the patch is applied.

Mention may be made, for example, of:
controlled-release patches with a hydrophobic polymeric matrix (FR 2 738 744 L'Oreal);
reservoir-type patches containing a reservoir of active substance, a diffusion membrane and an adhesive layer;
optimized-adhesion patches comprising a layer of hydrophobic polymer attached to a support layer and containing particles of active compound, particles of oil and particles of a water absorber (FR 2 761 889 L'Oreal);
   and advantageously the “reservoir” systems which allow controlled release of said active substance.

The patch generally comprises at least one polymeric matrix with a surface which is adhesive or able to become so, in particular after wetting, and is intended to be placed in contact with the skin.

The term “polymeric matrix” is intended to mean a layer of hydrophobic or hydrophilic polymer which may consist of a matrix which is self-adhesive (on dry skin and/or on wet skin).

The “hydrophobic” polymeric matrices constituting “conventional” patches are based in particular on polyacrylic or polyvinyl adhesive, on a silicone, polyurethane, styrene or isoprene polymer, whose crosslinking is preferably partial, so as to provide it with adhesion without the need for an additional adhesive layer. It is also possible to use an adhesive matrix made of latex, butyl or any other elastomeric adhesive.

The surface of the matrix intended to come into contact with the skin may be smooth or may have roughness or ridges.

The polymeric matrix is preferably deposited on a support.

The support may be an “occlusive” support. By way of example, the support consists of a thermoplastic material, chosen from high-density and low-density polyethylenes, polypropylenes, polyvinyl chlorides, ethylene-vinyl acetate copolymers, polyesters and polyurethanes, or of a complex of such materials. These materials may also be present in layered form with at least one metal foil such as an aluminium foil.

The support layer may be of any appropriate thickness that will provide the desired support and protection functions. Preferably, the thickness of the support layer is between approximately 20 μm and approximately 1.5 mm. Advantageously, the support layer is sufficiently flexible to be able to conform perfectly to the profile of the skin without causing any sensation of discomfort to the user.

Preferably, however, the support is “non-occlusive”. In the latter case, a support consisting of paper, of a porous or perforated thermoplastic material, of a woven, of a non-woven or of a perforated non-woven is advantageously used.

The patch preferably comprises at least one protective sheet which can be peeled off before application of the patch.

The patch may be packaged in a carton or in a protective envelope formed of two sheets of an impervious plastic paper/film complex, the paper being coated with a cold-seal adhesive and the sheets being sealed around the patch by contact of the adhesive-coated phases.

### Stimulation Induced by an Electrical Device

Devices which generate electrical stimulations, or electrostimulations, are conventionally used to act on the muscle tone, by muscle contraction. This muscle contraction may have the effect of generating mechanical stresses locally.

Electrostimulation uses a discontinuous low-voltage current which is transmitted by electrodes connected to an apparatus.

Electrostimulators generally comprise electrodes with a surface applied to the skin in the areas to be treated, said electrodes being connected to an electrical generator which applies electrical stimulations of varying frequencies ranging from 1 to 100 Hz, in particular from 0.2 to 60 Hz, and preferably from 0.2 to 20 Hz.

An example that may be mentioned as stimulation in the electrical form is a system for stimulating microdermal tension of the skin such as that described in application WO 06/116728. The output pulses can be adjusted within a range of approximately from 0.3 to 8 Hertz.

### Stimulation Induced by an Ultrasound or Sonophoresis Device

Ultrasound frequencies vary from 20 kHz to 10 MHz.

Use will be made generally of low-frequency (for example: 20 kHz-300 kHz) or medium-frequency (for example: 300 kHz-3 MHz) devices which are known to have an effect of muscle massage, of stimulation of the microcirculation and of cell contraction.

As an example of a device for stimulation in the form of ultrasound, mention may be made of a stimulator which applies to the skin a combined stimulus of ultrasound and low frequencies, such as that described in application WO 06/101295.

Mention may also, for example, be made of the Sonic Peeler® sold by the company Neps Inc., which, when applied to the skin, generates the ultrasonic vibrations.

Mention may also be made of the device described in application US 20050191252, which combines an ultrasound stimulation of the order of 5-6 MHz with an electrical stimulation of iontophoresis type.

According to a specific embodiment of the invention, it will also be possible to use, via at least one tensing device, a combination of these various types of stimulation in order to improve and/or optimize the application and/or the maintenance of mechanical stresses to and/or on the skin.

Preferably, the same tensing device will induce several types of stimulation.

Use may thus be made of a tensing device generating:
a mechanical stimulation and an electrical stimulation (for example: electrostimulating patches);
an electrical stimulation and an ultrasonic stimulation;
a mechanical stimulation and an ultrasonic stimulation.

According to a specific embodiment of the invention, the composition comprising at least one saccharide compound which increases the expression of mechanoreceptors in skin cells is contained in a reservoir of said tensing device and is released at the time said device is applied to the skin.

The invention also relates to a cosmetic kit comprising at least:
one composition comprising at least one saccharide compound which increases the expression of mechanoreceptors in skin cells; and
a tensing device intended to apply and/or maintain, in a controlled manner, mechanical stresses to and/or on the skin.

In particular, the tensing device intended to apply and/or maintain, in a controlled manner, mechanical stresses to and/or on the skin is chosen from a device which induces a mechanical stimulation, an electrical stimulation, an ultrasonic stimulation, and combinations thereof.

In particular, the tensing device intended to apply and/or maintain, in a controlled manner, mechanical stresses to and/or on the skin, of this kit, is a massage instrument chosen from a manual massage instrument or a massage instrument that requires an outside energy supply.

According to an alternative, the tensing device intended to maintain tensions on the skin, of this kit, is a support, in particular an adhesive support, capable of maintaining a tension on the skin.

A preferred support that can be used in the kit according to the invention is a patch.

In particular, the support, in particular adhesive support, has an elastic modulus ranging from 500 MPa to 10 000 MPa, preferably from 500 MPa to 2000 MPa.

According to a specific kit of the invention, the composition is contained in a reservoir of said tensing device intended to apply and/or maintain, in a controlled manner, mechanical stresses to and/or on the skin.

Examples of saccharide compounds which increase the expression of mechanoreceptors, present in the composition, are described hereinafter.

In particular, use will be made, in the composition constituting the care kit according to the invention, of a C-glycoside derivative which increases the expression of mechanoreceptors, in particular integrins, as described hereinafter.

According to a specific embodiment, the kit according to the invention may also comprise, firstly, (a) at least one composition comprising a saccharide compound which increases the expression of mechanoreceptors, and at least one tensing agent and, secondly, (b) at least one tensing device intended to apply and/or maintain, in a controlled manner, mechanical stresses to and/or on the skin.

Saccharide Compound which Increases the Expression of Mechanoreceptors

The expression “saccharide compound which increases the expression of mechanoreceptors in skin cells” is intended to mean in particular, according to the invention, any saccharide compound capable of inducing and/or stimulating the expression of mechanoreceptors in skin cells, in particular in the cells of the epidermis and of the dermis (for example: keratinocytes, fibroblasts).

Preferably, interest is focused on the saccharide compounds which increase the expression of integrins, and in particular the saccharide compounds which increase the expression of β1 integrins.

Such saccharide compounds can be selected according to conventional methods of detection by immunofluorescence or by quantatitive RT-PCR. Preferably, quantitative RT-PCR technology will be used.

The principle of detection by immunofluorescence consists in bringing cells in culture into contact with the saccharide compounds to be tested, and then in revealing the effect of said saccharide compounds on the expression of mechanoreceptors, and in particular integrins (for example: β1 integrins), using anti-integrin antibodies and secondary antibodies coupled to a fluorescent label (fluorescein).

The general principle of quantitative RT-PCR technology, which is preferred according to the invention, comprises, for example, the following steps:
the concentrations of the saccharide compounds to be tested are selected based on a study of cytotoxicity under the assay conditions;
human keratinocytes and/or fibroblasts are cultured in a culture medium suitable for these various cells types;
the culture medium is replaced with the same medium containing or not containing (control) the saccharide compound to be tested at the various concentrations selected;
after incubation for 24 h, for example, the mRNA is extracted and the traces of DNA are removed by treatment with DNAse, which is then inactivated;
a reverse transcription reaction is then carried out, followed by a quantification, by fluorescence, of the synthesized cDNA;
a first series of Q-PCRs is then carried out on the β-actin marker (control) in order to verify the homogeneity of the preparations to be compared;
Q-PCRs are then carried out in triplicate using pairs of primers specific for the β-actin sequences, and for markers that are specific for mechanoreceptors, and in particular integrins (for example: β1 integrins);
the differential expression of the integrins is then evaluated by an analysis of fluorescence in the amplified DNA;
the saccharide compounds for which an increase in the intensity of fluorescence is obtained, corresponding to a stimulation and/or an increase in the expression of integrins relative to the control condition (not treated with the agent), are selected.

The PCR (polymerase chain reaction) reactions can in particular be carried out by quantitative PCR with the “Light Cycler” system (Roche Molecular Systems Inc.) and according to the procedures recommended by the supplier.

Preferably, the saccharide compound which increases the expression of mechanoreceptors in skin cells, present in the composition, is a saccharide compound which increases the expression of integrins in skin cells.

The saccharide compounds according to the invention are low-molecular-weight saccharide compounds, in particular capable of diffusing in and/or penetrating the layers of the epidermis in order to increase the expression of mechanoreceptors.

According to the invention, the expression “low-molecular-weight saccharide compounds” is intended to mean saccharide compounds of molecular weight less than 100 kD, in particular less than 80 kD, even better still less than 60 kD, preferably less than 40 kD, even more preferably less than 20 kD, or even less than 10 kD.

According to a specific embodiment, the saccharide compounds according to the invention will have a molecular weight ranging from 0.01 to 10 kDa.

Compounds that are therefore excluded, as saccharide compounds which increase the expression of mechanoreceptors, according to the invention, are high-molecular-weight polysaccharides, i.e. polysaccharides of molecular weight greater than or equal to 100 kD with a gelling capacity which, by virtue of their large size, will remain at the surface of the skin.

Among the high-molecular-weight saccharides compounds, generally ranging from 100 kD to 10 000 kD, mention may in particular be made of gum arabic, gum ghatti, karaya gum, carob gum, guar gum, tamarind gum, xanthan, gellan, pectins, tragacanth, agar, alginate, carrageenan, furcelleran, konjac and cellulose derivatives.

The term “gelling capacity” is used to define the fact that, at a concentration of greater than or equal to 1% by weight in water, the viscosity of the solutions thus obtained is greater than or equal to 0.5 Pa·s for a shear rate equal to 1 s⁻¹. The measurements are carried out at 25° C. using a Haake RheoStress RS150 rheometer in the cone-plate configuration, the measurements of the measuring cone being: diameter: 60 mm and angle: 2°.

Preferably, the saccharide compounds which increase the expression of mechanoreceptors according to the invention will be chosen from: monosaccharides, polysaccharides and derivatives thereof, in particular the C-glycoside derivatives of monosaccharides or of polysaccharides.

The definition of the saccharide compounds according to the invention can extend to plant extracts containing them and/or to fractions of plant extracts enriched in these saccharide compounds, such as in particular extracts and/or fractions of wheat or of rye, and in particular of rye.

Monosaccharides

According to the invention, the term “monosaccharide” is intended to mean one saccharide unit, said monosaccharide having at least one hydroxyl function which must be free and/or, optionally, one or more optionally protected amine functions.

Advantageously, the monosaccharides may be in pyranose and/or furanose form, and of the L and/or D series, and chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine and N-acetyl-D-galactosamine.

Polysaccharides

The term “polysaccharide” is intended to mean a saccharide compound generally containing from 2 to 20 saccharide units, as described above, said polysaccharide having at least one hydroxyl function which must be free and/or, optionally, one or more optionally protected amine functions.

When the polysaccharide contains from 3 to 6 sugar units, it will be referred to as an oligosaccharide.

A distinction is made between homopolysaccharides consisting of the same monosaccharide and heteropolysaccharides made up of various monosaccharides.

In particular, the polysaccharide contains from 2 to 20 saccharide units, in particular from 5 to 20 saccharide units.

The polysaccharides according to the invention may be of linear and/or branched type.

They may be oligomers of monosaccharides, also called oligosaccharides, or polymers combining various monosaccharides, in pyranose and/or furanose form, and of the L and/or D series.

The polysaccharides according to the invention are advantageously chosen from: polysaccharides containing up to 20 saccharide units, chosen from D-maltose, D-lactose, D-cellobiose and D-maltotriose; a disaccharide combining a uronic acid, chosen from D-iduronic acid and D-glucuronic acid, with a hexosamine chosen from D-galactosamine, D-glucosamine, N-acetyl-D-galactosamine and N-acetyl-D-glucosamine; an oligosaccharide containing at least one xylose, advantageously chosen from xylobiose, methyl-β-xylobioside, xylotriose, xylotetraose, xylopentaose and xylohexaose, and preferably xylobiose, which is composed of two xylose molecules linked by a 1-4 linkage; or polysaccharides consisting of a chain of monosaccharides in pyranose form onto which are grafted saccharides units in furanose form, or vice versa, such as xylans and derivatives thereof.

As xylan derivatives, mention may, for example, be made of arabinoxylan, arabinoglucoxylan, galactogalactoglucoxylan, fucoglucoxylan, galactoarabinoxylan, galactoglucomannoxylan, galactoglucoxylan, galactomannoglucoxylan, galactoxylan, glucogalactoxylan, glucomannofucoxylan, glucoarabinoxylan, glucomannoxylan and glucoxylan.

A preferred xylan derivative is the arabinoxylan represented below:

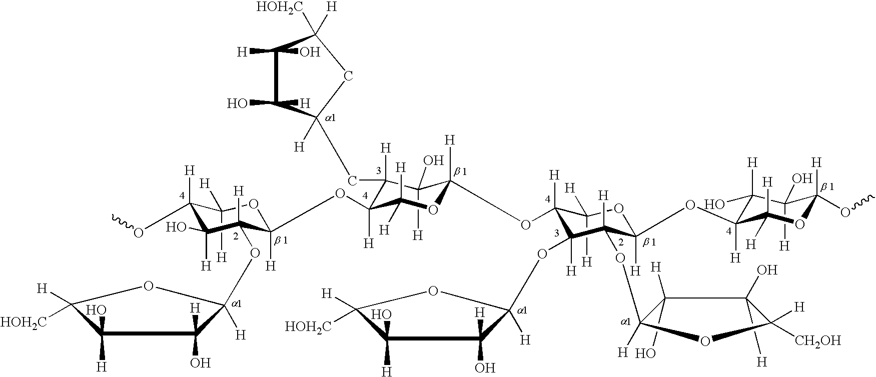

Arabinoxylans consist of a backbone of D-xylopyranose to which are linked α-L-arabinofuranose groups in a β-(1-4) linkage.

Use may also be made of plant extracts and/or fractions of extracts rich in at least one saccharide compound as defined above, and in particular plant extracts and/or fractions of extracts rich in a mixture of saccharide compounds as defined above. As preferred saccharide compounds, mention will be made of glucose, xylose, xylan and derivatives thereof.

By way of example, extracts and/or fractions containing at least glucose, xylose and an arabinoxylan, for instance extracts of rye or wheat, in particular rye, grain, may be used. Such extracts are in particular sold by Silab under the name Coheliss©.

As another example of commercialized rye grain extracts, mention may be made of Herbasol Extract Rye® from Cosmetochem.

c-Glycosides and Derivatives Thereof.

As saccharide compound which increases the expression of mechanoreceptors in skin cells, mention may also be made of C-glycoside derivatives such as those described in application WO 02/051828, incorporated herein by way of reference.

In particular, the C-glycoside derivative corresponds to general formula (I):

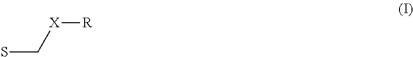

in which:
X represents a radical chosen from the groups:

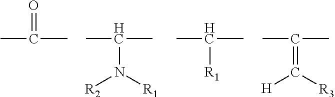

with R₁, R₂ and R₃ representing, independently of one another, a hydrogen atom, an OH group or a radical R, with R representing:
a saturated or unsaturated, linear, branched or cyclic C₁ to C₂₀ alkyl radical,
a saturated or unsaturated, linear, branched or cyclic C₁ to C₂₀ polyfluoroalkyl or perfluoroalkyl radical, or
a C₅ to C₂₀ aryl, in particular phenyl or alkylaryl radical, in particular benzyl,
   it being possible for the hydrocarbon-based chain constituting said radicals to be, where appropriate, interrupted with 1, 2, 3 or more heteroatoms chosen from:
oxygen,
sulphur,
nitrogen, and
silicon,
   and to be optionally substituted with at least one radical chosen from:
—OR₄,
—SR₄,
—NR₄R₅,
—COOR₄,
—CONHR₄,
CN,
a halogen atom,
a C₁ to C₆ polyfluoroalkyl or perfluoroalkyl radical,
a C₃ to C₈ cycloalkyl or heterocycloalkyl radical, and
a C₅ to C₁₈ aryl radical; optionally substituted,
   with R₄ and R₅ possibly representing, independently of one another, a hydrogen atom, a hydroxyl radical or a saturated or unsaturated, linear or branched C₁ to C₃₀, in particular C₁ to C₁₂, alkyl, acyl, perfluoroalkyl or polyfluoroalkyl radical,
S represents a monosaccharide or a polysaccharide containing up to 20 sugar units, in particular up to 6 sugar units, in pyranose and/or furanose form and of the L and/or D series, it being possible for said monosaccharide or polysaccharide to be substituted with a (CH₂)—OR₆ radical, with R₆ representing a hydrogen atom or a C₁ to C₆ alkyl radical, with a hydroxyl group and/or with an O-glycoside radical, and having at least one free hydroxyl function and/or an optionally protected amine function, and
the S—C bond represents a bond of C-anomeric nature, or a salt or isomer thereof.

In the context of the present invention, the term “halogen” means chlorine, fluorine, bromine or iodine.

The term “aryl” denotes an aromatic ring such as phenyl, optionally substituted with one or more C₁-C₄ alkyl radicals.

The term “C₃-C₈ cycloalkyl” denotes an aliphatic ring containing from 3 to 8 carbon atoms, including, for example, cyclopropyl, cyclopentyl and cyclohexyl.

Among the alkyl groups that are suitable for use in the invention, mention may be made especially of methyl, ethyl, isopropyl, n-propyl, n-butyl, t-butyl, isobutyl, sec-butyl, pentyl, n-hexyl, cyclopropyl, cyclopentyl, cyclohexyl and allyl groups.

According to one embodiment of the invention, it is possible to use a C-glycoside derivative corresponding to formula (I) for which S may represent a monosaccharide or a polysaccharide containing up to 6 sugar units, in pyranose and/or furanose form and of L and/or D series, the said mono- or polysaccharide bearing at least one mandatorily free hydroxyl function and/or optionally one or more mandatorily protected amine functions, X and R otherwise conserving all the definitions given previously.

Advantageously, the monosaccharide represented by S in formula (I) is chosen from D-glucose, D-galactose, D-mannose, D-xylose, D-lyxose, L-fucose, D-fucose, D-maltose, L-rhamnose, D-glucuronic acid, D-galacturonic acid, D-iduronic acid, N-acetyl-D-glucosamine, N-acetyl-D-galactosamine; and representes advantageously D-glucose, D-xylose, N-acetyl-D-glucosamine or L-fucose, and in particular D-xylose.

As regards the polysaccharides, they may contain up to 6 sugar units and are in particular chosen from D-maltose, D-lactose, D-cellobiose and D-maltotriose, a disaccharide combining a uronic acid, chosen from D-iduronic acid or D-glucuronic acid, with a hexosamine chosen from D-galactosamine, D-glucosamine, N-acetyl-D-galactosamine and N-acetyl-D-glucosamine, and an oligosaccharide containing at least one xylose advantageously chosen from xylobiose, methyl-β-xylobioside, xylotriose, xylotetraose, xylopentaose and xylohexaose, and preferably xylobiose, which is composed of two xylose molecules linked by a 1-4 linkage.

More particularly, S represents a monosaccharide chosen from D-glucose, D-xylose, D-fucose, D-galactose and D-maltose, and preferably D-xylose.

According to another embodiment of the invention, use may be made of C-glycoside derivatives corresponding to formula (I) for which X represents a group chosen from —CO—, —CH(OH)—, —CH(NR₁R₂)— and —CH(R)—, in particular —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferably a —CH(OH)— group, S and R conserving, moreover, all the definitions given above.

According to another embodiment of the invention, use may be made of a C-glycoside derivative corresponding to formula (I) for which R represents a saturated C₁ to C₂₀, in particular C₁ to C₁₀, or unsaturated C₂ to C₂₀, in particular C₂ to C₁₀, linear alkyl radical, or a saturated or unsaturated, branched or cyclic C₃ to C₂₀, in particular C₃ to C₁₀, alkyl radical, and which is optionally substituted as described above, S and R conserving, moreover, all the definitions given above. Preferably, R denotes a C₁-C₄, in particular C₁-C₃, linear radical optionally substituted with —OH, —COOH or —COOR₁₂, R₁₂ being a C₁-C₄ saturated alkyl radical, in particular ethyl. Preferably, R denotes an unsubstituted C₁-C₄, in particular C₁-C₂, linear alkyl radical, in particular ethyl.

Among the C-glycoside derivatives of formula (I), use is preferably made of those for which:
R represents a saturated C₁ to C₂₀, in particular C₁ to C₁₀, or unsaturated C₂ to C₂₀, in particular C₂ to C₁₀, linear alkyl radical, or a saturated or unsaturated, branched or cyclic C₃ to C₂₀, in particular C₃ to C₁₀ alkyl radical, and which is optionally substituted as described above;
S represents a monosaccharide as described above;
X represents —CO—, —CH(OH)—, —CH(NR₁R₂)— or —CH(R)— as described above.

Preferably, use is made of a C-glycoside derivative of formula (I) for which:
R denotes a C₁-C₄, in particular C₁-C₃, linear radical optionally substituted with —OH, —COOH or —COOR₁₂, R₁₂ being a saturated C₁-C₄ alkyl radical, in particular ethyl;
S represents a monosaccharide as described above;
X represents a group chosen from —CO—, —CH(OH)—, —CH(NH₂)—, —CH(NHCH₂CH₂CH₂OH)—, —CH(NHPh)- and —CH(CH₃)—, and more particularly a —CO—, —CH(OH)— or —CH(NH₂)— group, and preferably a —CH(OH)— group.

Preferably, use is made of a C-glycoside derivative of formula (I) for which:
R denotes an unsubstituted C₁-C₄, in particular C₁-C₂, linear alkyl radical, in particular ethyl;
S represents a monosaccharide as described above; in particular, D-glucose, D-xylose, N-acetyl-D-glucosamine or L-fucose, and in particular D-xylose;
X represents a group chosen from —CO—, —CH(OH)— and —CH(NH₂)—, and preferably a —CH(OH)— group.

The salts which are acceptable for the non-therapeutic use of the compounds described in the present invention include the conventional non-toxic salts of said compounds, such as those formed from organic or inorganic acids. By way of example, mention may be made of the salts of inorganic acids, such as sulphuric acid, hydrochloric acid, hydrobromic acid, hydroiodic acid, phosphoric acid or boric acid. Mention may also be made of the salts of organic acids, which may contain one or more carboxylic, sulphonic or phosphonic acid groups. They may be linear, branched or cyclic aliphatic acids or else aromatic acids. These acids may also contain one or more heteroatoms chosen from O and N, for example in the form of hydroxyl groups. Mention may in particular be made of propionic acid, acetic acid, terephthalic acid, citric acid and tartaric acid.

When the compound of formula (I) contains an acid group, the neutralization of the acid group(s) can be carried out with an inorganic base, such as LiOH, NaOH, KOH, Ca(OH)₂, NH₄OH, Mg(OH)₂ or Zn(OH)₂; with an organic base, such as a primary, secondary or tertiary alkylamine, for example triethylamine or butylamine. This primary, secondary or tertiary alkylamine may contain one or more nitrogen and/or oxygen atoms and may therefore contain, for example, one or more alcohol functions; mention may in particular be made of 2-amino-2-methylpropanol, triethanolamine, 2-dimethylaminopropanol and 2-amino-2-(hydroxymethyl)-1,3-propanediol. Mention may also be made of lysine or 3-(dimethylamino)propylamine.

The solvates that are acceptable for the compounds described in the present invention include conventional solvates such as those formed during the final step of preparation of said compounds due to the presence of solvents. By way of example, mention may be made of the solvates due to the presence of water or of linear or branched alcohols such as ethanol or isopropanol.

According to a preferred embodiment, use may be made, in the composition of the invention, of a C-glycoside derivative corresponding to formula (II):

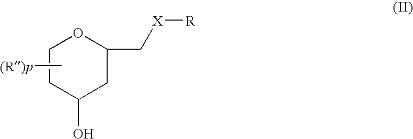

in which:
p represents an integer chosen from 2 and 3,
R and X are as defined above, and
R″ represents:
a radical (CH₂)—OR₆, with R₆ representing a hydrogen atom or a C₁ to C₆, in particular C₁ to C₄, alkyl radical,
a hydroxyl group, and/or
an O-glycoside radical.

The C-anomeric bond in formulae (I) and (II) may be α or β.

Preferably, X represents, in formulae (I) and (II), a function:

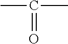

or a unit:

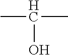

According to another specific embodiment, R represents therein a saturated, linear or branched C₁ to C₆ alkyl radical, and in particular a methyl radical.

The invention also extends to the optical and/or geometric isomers of the compounds of formulae (I) and (II), alone or as a mixture in all proportions, and also to the physiologically acceptable salts of these compounds.

The C-glycoside derivatives in accordance with the invention may be used alone or as a mixture and in any proportion.

For the purpose of the present invention, the term “mixture” relates to mixtures of the various isomeric forms of the same compound, just as it does to mixtures of various compounds of general formula I and/or of their respective isomeric forms.

The C-glycoside derivatives may be of natural or synthetic origin, completely or partially purified from any preparation containing them.

The term “natural origin” is intended to mean a derivative extracted from a natural material such as a plant, for example. The term “synthetic origin” is intended to mean a derivative prepared by chemical synthesis or by biotechnology.

The expression “completely or partially purified” signifies herein that, during its synthesis or relative to its natural state (fresh or dried plant or cells), the compounds of formulae (I) and (II) according to the invention have been concentrated and/or freed, respectively, of at least part of the side-reaction products derived from their synthesis or of at least part of the other constituents of the natural material in which they are present.

In addition, certain hydroxyl functions of the C-glycoside derivatives of formula (II) may be sulphated after selective protection of the other hydroxyl functions. This sulphation reaction after protection is in particular described by A. Lubineau in the reference J. Chem. Soc. Chem. Commun., 1993, page 1419.

By way of non-limiting illustration of the C-glycoside derivatives that can be used in the composition of the invention, mention may in particular be made of the following compounds:
C-β-D-xylopyranoside-n-propan-2-one,
C-α-D-xylopyranoside-n-propan-2-one,
C-β-D-(3,4,5-triacetoxy)xylopyranoside-n-propan-2-one,
C-β-D-xylopyranoside-2-hydroxypropane,
C-α-D-xylopyranoside-2-hydroxypropane,
1-(C-β-D-fucopyranoside)propan-2-one,
1-(C-α-D-fucopyranoside)propan-2-one,
1-(C-β-L-fucopyranoside)propan-2-one,
1-(C-α-L-fucopyranoside)propan-2-one,
1-(C-β-D-fucopyranoside)-2-hydroxypropane,
1-(C-α-D-fucopyranoside)-2-hydroxypropane,
1-(C-β-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-g lucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl)propan-2-one,
1-(C-α-D-fucofuranosyl)propan-2-one,
1-(C-1-L-fucofuranosyl)propan-2-one,
1-(C-α-L-fucofuranosyl)propan-2-one,
C-β-D-maltopyranoside-n-propan-2-one,
C-α-D-maltopyranoside-n-propan-2-one
C-β-D-maltopyranoside-2-hydroxypropane,
C-α-D-maltopyranoside-2-hydroxypropane,
   isomers thereof and mixtures thereof.

These C-glycoside derivatives can increase the expression of mechanoreceptors in skin cells by inducing the activation of intracellular signals which lead to transactivation of the integrin promoter and to their expression.

Advantageously, the C-glycosides according to the invention are chosen from:
C-β-D-xylopyranoside-n-propan-2-one
C-β-D-(3,4,5-triacetoxy)xylopyranoside-n-propan-2-one
C-β-D-xylopyranoside-2-hydroxypropan-2-one and derivatives thereof.

According to one embodiment, C-β-D-xylopyranoside-2-hydroxypropane or C-α-D-xylopyranoside-2-hydroxypropane, and better still C-β-D-xylopyranoside-2-hydroxypropane, can be advantageously used for the preparation of a composition according to the invention.

According to a specific embodiment, the C-glycoside derivative is C-β-D-xylopyranoside-2-hydroxypropane in the form of a solution at 30% by weight of active material in a water/propylene glycol mixture (60/40% by weight), such as the product manufactured by CHIMEX under the trade name “MEXORYL SBB®”.

The saccharide compounds that can be used in the compositions of the invention may be of natural or synthetic origin.

The term “natural origin” is intended to mean a derivative extracted from a natural material such as a plant, for example. The term “synthetic origin” is intended to mean a derivative prepared by chemical synthesis or by biotechnology.

As particularly preferred saccharide compounds which increase the expression of the mechanoreceptors, it is possible to use arabinoxylans and C-glycosides of formula (I) for which
R denotes a C₁-C₄ and especially C₁-C₂ unsubstituted linear alkyl radical, in particular ethyl;
S represents a monosaccharide as described previously; especially D-glucose, D-xylose, N-acetyl-D-glucosamine or L-fucose, and in particular D-xylose;
X represents a group chosen from —CO—, —CH(OH)— and —CH(NH₂)—, and preferentially a group —CH(OH)—.
   Arabinoxylans are preferably used as saccharide compound.

C-β-D-Xylopyranoside-2-hydroxypropane is also preferred.

The amount of saccharide compound which increases the expression of mechanoreceptors, that can be used in the compositions according to the invention, depends of course on the desired effect and should be an amount that is effective for inducing and/or increasing the expression of mechanoreceptors in skin cells.

To give an order of magnitude, the composition of the invention may contain at least one saccharide compound which increases the expression of mechanoreceptors, in an amount representing from 0.00001% to 30% of the total weight of the composition, in particular in an amount representing from 0.0001% to 10%, and even more preferably in an amount ranging from 0.01% to 5% by weight relative to the total weight of the composition.

According to a specific embodiment of the invention, when the tensing agent and the saccharide compound are in the same composition, and the tensing agent is a polysaccharide containing at least one rhamnose, said saccharide compound which increases the expression of mechanoreceptors will not contain rhamnose.

A preferred composition that may be used is a composition comprising:

a tensing agent chosen from interpenetrating polymers comprising a polyurethane and an acrylic polymer;
and a saccharide compound which increases the expression of the mechanoreceptors, chosen from arabinoxylans and C-glycosides of formula (I) for which
R denotes a C₁-C₄ and especially C₁-C₂ unsubstituted linear alkyl radical, in particular ethyl;
S represents a monosaccharide as described previously; especially D-glucose, D-xylose, N-acetyl-D-glucosamine or L-fucose, and in particular D-xylose;
X represents a group chosen from —CO—, —CH(OH)— and —CH(NH₂)—, and preferentially a group —CH(OH)—.

The composition may preferably comprise a tensing agent chosen from interpenetrating polymers comprising a polyurethane and an acrylic polymer; and a saccharide compound which increases the expression of the mechanoreceptors, chosen from arabinoxylans.

The composition may preferably comprise a tensing agent chosen from interpenetrating polymers comprising a polyurethane and an acrylic polymer; and C-β-D-xylopyranoside-2-hydroxypropane.

The composition according to the invention comprises a physiologically acceptable medium, i.e. a medium that is compatible with the skin of the face and/or the body. It is preferably a cosmetically acceptable medium, i.e. a medium which has a pleasant colour, odour and feel, and which does not generate any unacceptable discomfort (stinging, tautness, redness) liable to dissuade consumers from using this composition.

The composition according to the invention may be a body or facial care composition, or a makeup composition.

The composition according to the invention may be in any of the pharmaceutical forms conventionally used for topical application, and in particular in the form of dispersions of the aqueous lotion or gel type, emulsions with a liquid or semi-liquid consistency of the milk type, obtained by dispersion of a fatty phase in an aqueous phase (O/W) or conversely (W/O), or suspensions or emulsions with a soft, semi-solid or solid consistency, of the cream or gel type, or in the form of a serum, a stick or alternatively multiple emulsions (W/O/W or O/W/O), microemulsions, vesicular dispersions of ionic and/or non-ionic type, or wax/aqueous phase dispersions. These compositions are prepared according to the usual methods.

When the composition that can be used according to the invention is an emulsion, the proportion of the fatty phase may range from 5% to 80% by weight, and preferably from 5% to 50% by weight, relative to the total weight of the composition. The oils, the waxes, the emulsifiers and the coemulsifiers used in the composition in the form of an emulsion are chosen from those conventionally used in the cosmetics field. The emulsifier and the coemulsifier are present, in the composition, in a proportion ranging from 0.3% to 30% by weight, and preferably from 0.5% to 20% by weight, relative to the total weight of the composition. The emulsion may also contain lipid vesicles.

When the composition that can be used according to the invention is an oily solution or gel, the fatty phase may represent more than 90% of the total weight of the composition.

In a known manner, the composition of the invention may also contain the usual adjuvants in the cosmetics and dermatological fields, such as hydrophilic or lipophilic gelling agents, hydrophilic or lipophilic active agents, preserving agents, antioxidants, solvents, fragrances, fillers, screening agents, pigments, chelating agents, odour absorbers and dyestuffs. The amounts of these various adjuvants are those conventionally used in the fields under consideration, representing for example from 0.01% to 20% of the total weight of the composition. Depending on their nature, these adjuvants may be introduced into the fatty phase, into the aqueous phase, into lipid vesicles and/or into nanoparticles.

When the composition of the invention is an emulsion, the proportion of the fatty phase may range from 5% to 80% by weight, and preferably from 5% to 50% of the total weight of the composition. The oils, the emulsifiers and the coemulsifiers used in the composition in the form of an emulsion are chosen from those conventionally used in the field under consideration. The emulsifier and the coemulsifier are present, in the composition, in a proportion ranging from 0.3% to 30% by weight, and preferably from 0.5% to 20% of the total weight of the composition.

As oils or waxes that can be used in the invention, mention may be made of mineral oils (liquid petroleum jelly), plant oils (liquid fraction of shea butter, sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

As emulsifiers that can be used in the invention, mention may, for example, be made of glyceryl stearate, polysorbate 60 and the mixture of PEG-6/PEG-32/glycol stearate sold under the name Tefose® 63 by the company Gattefosse.

As solvents that can be used in the invention, mention may be made of lower alcohols, in particular ethanol and isopropanol, and propylene glycol.

As emulsifiers and coemulsifiers that can be used in the invention, mention may, for example, be made of fatty acid esters of polyethylene glycol, such as PEG-40 stearate or PEG-100 stearate, and fatty acid esters of a polyol, such as glyceryl stearate and sorbitan tristearate.

As hydrophilic gelling agents that can be used in the invention, mention may be made of carboxyvinyl polymers (carbomer), acrylic copolymers such as acrylate/alkyl acrylate copolymers, polyacrylamides, polysaccharides such as hydroxypropylcellulose, natural gums and clays, and, as lipophilic gelling agents, mention may be made of modified clays such as bentones, metal salts of fatty acids, such as aluminium stearate, and hydrophobic silica, ethylcellulose and polyethylene.

Of course, those skilled in the art will take care to select this or these possible additional compound(s) and/or the amount thereof in such a way that the advantageous properties of the composition according to the invention are not, or are not substantially, impaired by the envisaged addition.

The application of the composition according to the invention is carried out according to usual techniques, for example by application of creams, gels, sera or lotions to the skin intended to be treated, in particular the skin of the body, face and/or neck.

The invention also relates to a skincare kit comprising at least:
a first composition comprising, in a physiologically acceptable medium, at least one saccharide compound which increases the expression of mechanoreceptors in skin cells;
a second composition comprising, in a physiologically acceptable medium, at least one tensing agent.

Examples of saccharide compounds which increase the expression of integrins are described previously in the description.

The tensing agent present in the second composition may be chosen from synthetic polymers, vegetable or animal proteins, polysaccharides of plant origin which may or may not be in the form of microgels, mixed silicates, colloidal particles of inorganic fillers, and mixtures thereof.

Examples of tensing agents that can be used according to the invention are described previously in the description.

The invention also relates to a process for the cosmetic treatment of the skin, comprising the application, via the use of at least one composition, of at least one saccharide compound which increases the expression of mechanoreceptors in skin cells and of at least one tensing agent.

The application can be carried out simultaneously (1 composition) or sequentially (2 separate compositions).

The term “sequential application” is intended to mean a successive (immediate) or delayed application.

In particular, the saccharide compound which increases the expression of mechanoreceptors in skin cells is chosen from a xylan derivative, C-glycosides and derivatives thereof.

According to a specific embodiment, the process according to the invention comprises the simultaneous or sequential application of at least one saccharide compound which increases the expression of mechanoreceptors in skin cells, of at least one tensing agent and/or of at least one tensing device intended to apply and/or maintain, in a controlled manner, mechanical stresses to and/or on the skin.

Examples of such tensing devices are described above.

The process according to the invention is intended in particular to promote skin homeostasis and/or to improve the mechanical properties of the skin (for example, firmness, elasticity, tonicity) and/or to improve skin density, and/or to promote regeneration and/or reorganization of the papillary dermis, and/or to promote regeneration and/or reorganization of the extracellular matrix, in addition to an immediate effect of smoothing out the microrelief of the skin and the wrinkles and fine lines, provided by the tensing agent and/or the tensing device.

According to a specific embodiment, the process for the cosmetic treatment of the skin comprises the sequential application of at least one composition comprising at least one saccharide compound which increases the expression of mechanoreceptors in skin cells, and a composition comprising at least one tensing agent.

For sequential application, the composition comprising the saccharide compound which increases the expression of mechanoreceptors in skin cells is preferably applied before the composition comprising the tensing agent during the first days of treatment.

The order of application of the compositions is relatively unimportant after a few days of treatment.

According to a specific embodiment, the composition(s) according to the invention may be applied on individuals with soft and/or flaccid skin or to areas of the body which show a loss of elasticity and/or of firmness and/or of tonicity.

In particular, the composition may be applied to the face, the stomach and the thighs.

Advantageously, and in order to obtain a lasting effect of the tensing agents, over time, on skin homeostasis, the application of the composition according to the invention or of the care kit may be carried out twice-weekly, and better still daily, in the morning and/or the evening.

The effect of repeated application to the skin of tensing agents according to the invention, on the mechanical properties of the skin and in particular on the firmness, the elasticity and the extensibility of the skin, may also be confirmed and/or evaluated in vivo using instrumental devices such as those described hereinafter.

### The Torquemeter:

This apparatus aims to measure the variations in extensibility, in firmness/elasticity and in tonicity of the skin. The apparatus imposes a torsion in the plane of the skin for a given period of time: the skin is then subjected to a stretching which corresponds to its extensibility (Ue); after torsion is stopped, the skin regains its initial “shape”, and thus its tonicity (Ur) is evaluated. The firmness/elasticity of the skin corresponds to the ratio Ur/Ue (tonicity over extensibility).

### The Cutometer:

The cutometer is a suction apparatus consisting of a cylinder of from 1 to 3 mm in diameter, which is applied to the skin. Drawing up the skin by means of a pump connected to the cylinder induces a vertical displacement of the skin, which makes it possible to evaluate the mechanical properties of the skin.

The Densiscore:

The densiscore is an apparatus for measuring skin density. It subjects the skin locally to a mechanical stress which gives rise to folds, the number and size of which are directly related to the skin density. Evaluation by trained experts of the profile of the skin subjected to the densiscore makes it possible to evaluate the skin density.

Moreover, the effect of repeated application to the skin of tensing agents according to the invention, on reorganization of the extracellular matrix, may also be confirmed and/or evaluated in vivo by means of an ultrasound echography technique.

The degradation and/or disorganization of the extracellular matrix at the level of the papillary dermis is a major cause of ageing of the skin. It is partly responsible for the appearance of wrinkles and for the loss of the density, firmness and extensibility of the skin. This degradation and/or disorganization of the extracellular matrix is even more visible in individuals who have mature (>40 years), or even very mature (>60-65 years) skin.

Ultrasound echography makes it possible to obtain 2D or 3D images of cutaneous tissues. The intensity of the echos reflected provides information on the nature, density and organization of the constituents of the dermis. In particular, it demonstrates differences between the superficial dermis or papillary dermis and the deep dermis or reticular dermis. This technique thus makes it possible to evaluate the effect of repeated application of the tensing agents according to the invention on the reorganization and restructuring of the papillary dermis.

In the context of this process, the composition may, for example, be a care composition or a makeup composition.

The invention also relates to the cosmetic use, in a composition comprising a physiologically acceptable medium, of at least one saccharide compound which increases the expression of mechanoreceptors in skin cells, as an agent intended to sensitize cells to the mechanical tensions induced by the topical application of a tensing agent.

In particular, the saccharide compound which increases the expression of mechanoreceptors in skin cells is intended to potentiate and/or prolong the biomechanical effect of a tensing agent applied topically to the skin, at the level of the cells of the epidermis and/or of the dermis, in particular the effect of the tensing agent on maintaining and/or increasing skin thickness, smoothing out wrinkles and fine lines and/or improving the mechanical properties of the skin.

The biomechanical effect of the tensing agent at the level of the cells of the epidermis and of the dermis is in particular defined by an improvement in skin homeostasis, in particular maintenance of and/or an increase in skin thickness, smoothing out of wrinkles and fine lines and/or an improvement in the mechanical properties of the skin (for example: firmness, elasticity, tonicity), and/or an improvement in skin density, and/or promoting regeneration and/or reorganization of the papillary dermis, and/or promoting regeneration and/or reorganization of the extracellular matrix.

In particular, the saccharide compound which increases the expression of mechanoreceptors in skin cells is intended to potentiate and/or prolong the effect of the tensing agent on the reduction of epidermal differentiation processes and/or the improvement of regeneration and/or renewal of the skin.

The invention therefore also relates to the cosmetic use, in a composition comprising a physiologically acceptable medium, of at least one saccharide compound which increases the expression of mechanoreceptors in skin cells, in combination with a tensing agent and/or a tensing device, or to the use of a composition or of a kit containing said combination, for promoting improved skin homeostasis, increased skin thickness, an improvement in the radiance of the complexion, skin density, regeneration and/or reorganization of the papillary dermis, regeneration and/or reorganization of the extracellular matrix and/or an improvement in the firmness, the elasticity and/or the tonicity of the skin.

According to a first embodiment, the saccharide compound which increases the expression of mechanoreceptors in skin cells and the tensing agent are present in the same composition.

According to an alternative, the saccharide compound which increases the expression of mechanoreceptors in skin cells and the tensing agent are packaged in two separate compositions.

The saccharide compounds which increase the expression of mechanoreceptors in skin cells and the tensing agents that can be used according to the present invention can be chosen from the examples of compounds described above in the description.

The present invention also relates to a cosmetic skincare process comprising the simultaneous or sequential application:
(i) of a composition comprising at least one saccharide compound which increases the expression of mechanoreceptors in skin cells;
(ii) of a tensing device intended to apply and/or maintain, in a controlled manner, mechanical stresses to and/or on the skin.

According to the invention, “simultaneous application” is intended to mean an embodiment in which the composition is contained in a reservoir of said device or else the composition is applied prior to said device before application of the latter to the skin. Preferably, the composition will be contained in said device.

The term “sequential application” is intended to mean an embodiment in which the composition and the device are applied to the skin successively (immediately) or in a manner delayed over time: preferably, the composition will be applied before the device at the time of the first application(s), the composition being intended to sensitize the cells to the mechanical stresses introduced by said device; the order of application is relatively unimportant during the subsequent applications, for a daily treatment at a rate of one or two applications per day.

In particular, the invention relates to a cosmetic skincare process comprising the simultaneous or sequential application:
(i) of a composition comprising at least one saccharide compound which increases the expression of mechanoreceptors in skin cells;
(ii) of a device intended to apply and/or maintain, in a controlled manner, a stress chosen from a tension, a traction, a pressure and combinations thereof.

The process according to the invention is intended in particular to promote skin homeostasis and/or to improve the mechanical properties of the skin (for example: firmness, elasticity, tonicity) and/or to promote the radiance of the complexion and/or to improve skin density and/or regeneration of the skin.

According to a specific embodiment, the composition and the tensing device according to the invention may be applied on individuals with a dull and/or poorly defined complexion in order to promote the radiance of the complexion.

According to another embodiment, the composition and the tensing device according to the invention may be applied on individuals with soft and/or flaccid skin or to areas of the body which show a loss of elasticity and/or of firmness.

In particular, the composition may be applied to the face, the stomach and the thighs.

The invention also relates to a process as described above, for combating skin ageing, characterized in that a tensing device intended to apply and/or maintain a traction and/or a tension to and/or on the skin is used.

The invention also relates to a process as described above, for combating skin ageing, characterized in that a tensing device intended to apply and/or maintain a pressure to and/or on the skin is used.

Advantageously, and in order to obtain a lasting effect, over time, on skin homeostasis, the application of the composition and of the tensing device according to the invention may be carried out twice-weekly, preferably daily, in the morning and/or the evening.

The process according to the invention may consist, for example, of a daily application, in the morning and/or evening, simultaneously or sequentially (successively or delayed over time), of the composition and of the tensing device.

The application is carried out on the areas of the face and/or body to be treated.

The tensing device intended to generate the mechanical stresses is applied to the skin for a period of preferably greater than or equal to 1 minute and which can range, for example, from several minutes (in particular in the case of a massage with a massage instrument) to 1 or more hours (for example, in the case of a patch for controlled release of the active agent).

The invention also relates to the joint use of a composition comprising at least one saccharide compound which increases the expression of mechanoreceptors in skin cells and of a tensing device intended to apply and/or maintain, in a controlled manner, mechanical stresses to and/or on the skin, said composition being intended to sensitize the cells of the skin to the mechanical stresses induced by the application of said tensing device.

This joint use has in particular the effect of potentiating and/or prolonging the effect of the mechanical stresses induced by the application of said tensing device on the improvement of skin thickness, the improvement of the radiance of the complexion, the improvement of the density and/or the improvement of the mechanical properties of the skin (firmness, elasticity, tonicity) and/or the improvement of the regeneration of the skin and/or its cicatrization.

The invention will now be described with reference to the following examples, given by way of non-limiting illustration. In these examples, unless otherwise indicated, the amounts are expressed as percentages by weight.

FIG. 1: example of a mosaic deposit of Hybridur 875 (magnification ×30).

FIG. 2: example of a curve of force as a function of displacement.

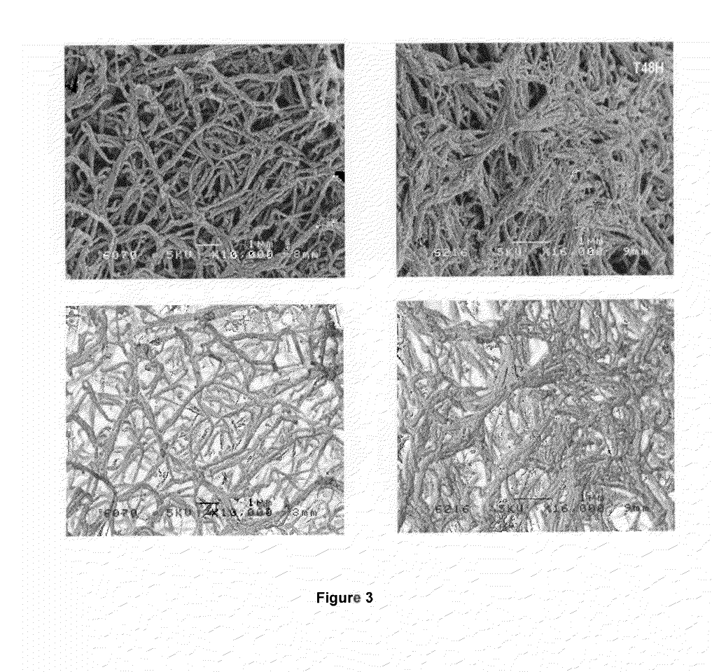

FIG. 3: schematic representation of an electron micrograph showing the effect of Hybridur 875 on the reorganization of the collagen fibrils in the extracellular matrix.

### EXAMPLES

### Example 1

### Demonstration of the Biological Effects of the Tensing Agents

a) Effect on Differential Gene Expression

The biological effects of the tensing agents were demonstrated after application to EPISKIN® reconstructed epiderma.

### Reconstructed Epidermis Culture Conditions

The EPISKIN® reconstructed epiderma used were obtained on D15. They were placed in a maintenance medium for 8 hours. They were then transferred into a DMEM/Ham F12 medium free from EGF, pituitary extract and foetal calf serum. The epidermes were equilibrated in this medium for 24 hours.

### Preparation of the Tensing Agent: Ethylenic Copolymer of Methyl Methacrylate/Methacrylic Acid Copolymer Type

### 1^{st} Step: Polymer Synthesis

1 g of Trigonox 21S (t-butylperoxy-2-ethyl hexanoate) and 200 g of methyl ethyl ketone were placed in a 2-l jacketed reactor. The mixture was refluxed for 1 h. After 1 h, a mixture of 170 g of methyl methacrylate and 30 g of methacrylic acid was added dropwise over a period of 1 h. The colourless mixture became viscous. The heating was interrupted 6 h after the addition of the monomers.

Composition by NMR: 85.1% methyl methacrylate, 14.9% methacrylic acid.

Mass by GC in THF (polystyrene standards): Mp=98772; Mn=61261; Mw=105698; Ip=1.7.

### 2^{nd} Step: Dispersion of the Polymer in Water

200 g of methyl ethyl ketone were added to the reaction medium above and the mixture was heated to 60° C. 30.86 g of 2-amino-2-methylpropanol and 1200 g of water were added dropwise. The volatile solvents were evaporated off by heating to 100° C. A transparent yellow aqueous dispersion was obtained.

One hundred microlitres of an aqueous dispersion of this ethylenic copolymer were then applied to the EPISKINs in this culture medium and left in contact with the epidermes for 24 hours in a chamber thermostated at 37° C. and 40% relative humidity. At the end of this period, the epidermes were removed and extracted for the cDNA array studies.

### Dedicated Minichip Analysis

The gene expression was analysed using standard DNA arrays, dedicated to research and suitable for screening. These minichips were prepared on a nylon substrate by attaching cDNAs specific for the markers involved in the regulation of keratinocyte physiology. The analysis was carried out by means of a specific miniaturized and optimized technology based on the use of mRNA and of labelling with phosphorus 33 (P33).

Schematically, the mRNA of the cells was extracted and purified using trireagent, and the mRNA of each culture was reverse transcribed using oligo (dT) and a P33-labelled deoxynucleotide triphosphate. Multiple labelled “target” cDNA sequences were therefore prepared for each EPISKIN® reconstructed epidermis. These targets were then hybridized, under optimized conditions, to the “probe” cDNAs in excess, attached to the membranes. After washing, the amount of labelled target was revealed by autoradiography and direct counting on a PhosphorImager. The analysis of the membranes was carried out using the Imagequant software.

The results are expressed in relative expression units. The levels of expression were corrected 1) with respect to the average background noise present on each membrane, and 2) with respect to the differences in intensity of labelling of the various probes used. This correction is carried out on the basis of the differences in labelling intensity of the reference genes. The mean of the counting results for “housekeeping gene” markers, the expression of which is generally considered to be stable, was taken as a reference for quantifying, relatively, the expression of the other markers.

The limit of significance was set at 180% of the non-treated control for a stimulant effect and at 50% of the control for a repressor effect.

### Results

### Modulation of the Expression of Genes Involved in Keratinocyte Differentiation:

25 genes among the 159 present on the dedicated minichips were modulated by the tensing agent. These genes were involved in the regulation of keratinocyte or fibroblast physiology.

The following table gives all the results obtained regarding the effect of the tensing agents on the expression of these genes.

| | | | | | % variation of |
|---|---|---|---|---|---|
| | | | | | the expression |
| | | | | Growth | of the genes |
| | | | | factors, | relative to the |
| | | Differentiation | Metallo- | cytokines, | non-treated |
| Abbreviation | Gene name | markers | proteinase | receptors | control |
| AZGP1 | alpha-2- | | | | 21 |
| | glycoprotein 1, | | | | |
| | zinc | | | | |
| B2M | beta-2- | | | | 486 |
| | microglobulin | | | | |
| CDSN | corneodesmosin | x | | | 51 |
| CST6 | cystatin 6 | x | | | 35 |
| CK1 | cytokeratin 1 | x | | | 980 |
| KRT19 | type I | x | | | 293 |
| | cytoskeletal | | | | |
| | keratin 19 | | | | |
| KRT2E | type II | x | | | 1163 |
| | cytoskeletal | | | | |
| | epidermal keratin | | | | |
| | 2 (KRT2E; | | | | |
| | KRT2A) | | | | |
| KRT6A | type II | x | | | 386 |
| | cytoskeletal | | | | |
| | keratin 6: K6A | | | | |
| | keratin (KRT6A) | | | | |
| LOR | loricrin | x | | | 50 |
| NICE-1 | NICE-1 protein | x | | | 58 |
| CRBP1 | retinol-binding | x | | | 326 |
| | protein I | | | | |
| SPRL1B, | SPRL “small | x | | | 20 |
| XP5 | proline rich-like” | | | | |
| | protein | | | | |
| | (epidermal | | | | |
| | differentiation | | | | |
| | complex) 1B or | | | | |
| | skin-specific | | | | |
| | protein (XP5) | | | | |
| SPRL6A, | small proline-rich- | x | | | 37 |
| LEP16 | like protein | | | | |
| | (epidermal | | | | |
| | differentiation | | | | |
| | complex) 6A or | | | | |
| | SPRL6A; or late | | | | |
| | envelope protein | | | | |
| | 16 | | | | |
| KLK7 | kallikrein 7 | x | | | 56 |
| SBS | suprabasin (SBS) | x | | | 33 |
| ZYX | zyxin | x | | | 53 |
| MMP3 | matrix | | x | | 37 |
| | metalloproteinase | | | | |
| | 3 (MMP3) | | | | |
| IL6 | interleukin-6 | | | x | 340 |
| IL8 | interleukin-8 | | | x | 259 |
| TLR1 | toll-like receptor 1 | | | x | 434 |
| TGFB1 | transforming | | | x | 437 |
| | growth factor | | | | |
| | beta 1 | | | | |
| HMOX1 | heme oxygenase 1 | | | | 60 |
| HSPCA | 90 kDa heat | | | | 1083 |
| | shock protein 1 | | | | |
| MT1H | metallothionein | | | | 52 |
| | IH | | | | |
| MIF | macrophage | | | | 54 |
| | migration | | | | |
| | inhibitory factor | | | | |

The ethylenic copolymer tested reduced the expression of several proteins which constitute the stratum corneum, such as corneodesmosin and loricrin by a factor of two, and suprabasin by a factor of 3, which suggests that the copolymer reduces the terminal differentiation process.

The acrylic copolymer increases, moreover, the expression of several cytoskeletal intermediate filament proteins, cytokeratins, which are found in particular in foetal epithelia and regenerative epithelia. After 24 h of treatment, the expression of cytokeratin 1 is increased by a factor of 10 and the expression of cytokeratin 19 is increased by a factor of 3. Although they are present in adult epidermis, these two cytokeratins have been described as being expressed in many types of epithelial tissues, in particular in non-stratified epithelia, and also foetal epithelia (Haake et al., Exp Cell Res., 1997 Feb. 25; 231 (1): 83-95). The expression of cytokeratin 2E/A is also increased, by a factor of 10: this cytokeratin 2 has been described as being expressed both in an adult epidermis and in a foetal epidermis. Finally, the expression of cytokeratin 6 is increased by a factor of 4. This cytokeratin 6 has been described as being overexpressed in regenerative epidermes, in particular during cicatrization (Mazzalupo et al., 2003 February; 226(2):356-65), which suggests that, over the course of the tensions provided by the application of the acrylic copolymer, the epidermes adopt characteristics of regenerative epidermes.

The ethylenic copolymer according to the invention reduces the expression of complexes required for the keratinocyte differentiation process, such as SPRL, also called LEP10, by a factor of 5, and SPRL6 by a factor of 2.

In parallel to this, these results show that the copolymer increases the expression of CRBP1, which is involved in the response of cells to retinol, by a factor 3, which suggests that the tensions may sensitize the cells to retinol.

### Modulation of TGFβ6 Expression

The expression of TGFβ is increased by a factor of 4. This cytokine increases the expression of all the fibrillar collagens, just as it does that of plasminogen activator type I, PAI1, and decreases the expression of several enzymes involved in the degradation of the extracellular matrix, metalloproteinases.

Over the course of the tensions generated by the acrylic latex, the TGFβ induced could diffuse in the dermis and thus induce tissue repair. The increase in TGFβ expression brought about by the tensions can be considered to be a control of the sensitivity of the cells to the tensions generated by the acrylic latex tensing agent.

### Decrease in the Expression of Metalloproteinases:

The ethylenic copolymer decreases the expression of metalloproteinase 3 which is involved, firstly, in cell migration and, secondly, in extracellular matrix degradation. The ethylenic copolymer thus inhibits extracellular matrix degradation and plays a role in cell migration.

### Decrease in Zyxin:

The ethylenic copolymer decreased the expression of zyxin, which is known to be located at the adhesion complexes and to play a role in cell morphology.

### Increase in the Cell Response to Environmental Stresses

The ethylenic copolymer increased the expression of the chaperone protein HSP90A by a factor of 10. The HSP90A proteins play a fundamental role during the protein maturation process. They regulate the conformation of kinase and of transcription factors and as a result control their activity and their degradation.

All these data show that the mechanical tensions applied via an effective amount of the tensing agent according to the invention are felt by the keratinocytes as a stimulus which leads to a slowing down of the epidermal differentiation process; the modulation of the gene expression mentioned above appears to show that the epidermis acquires moreover, a regenerative epidermal phenotype. These results indicate that topical application of an effective amount of at least one tensing agent makes it possible to promote skin homeostasis and thus to increase skin thickness and/or to improve the mechanical properties of the skin.

Moreover, the increase in HSP90 expression implies that the tensions will reinforce the ability of the epidermis to combat the impairment of skin homeostasis induced by environmental stresses.

### Example 2

### Tensing Agent Mechanical Strength Test

The mechanical strength test consists in applying compressive stress to breaking point to the tensing agent to be tested, at the surface of a flexible and deformable foam. The use of this foam support makes it possible to impose a considerable deformation on the tensing agent to be tested, deposited at the surface, and therefore allows its breaking strength to be quantified.

The substrate consists of a neoprene foam 13 mm thick.

The tensing agent which is soluble or dispersible in water at a temperature ranging from 25° C. to 50° C. at a concentration of 7% by weight in water or at the maximum concentration by weight at which it forms, in water at a temperature ranging from 25° C. to 50° C., a homogeneous medium visible to the naked eye, is deposited on this substrate so as to obtain, after drying for 24 h, a deposit with a thickness of 15 to 30 μm. The deposits were made using a film-drawing device depositing 650 μm wet.

The mechanical compressive stress is exerted by means of a cylindrical punch 1 mm in diameter; the rate of displacement of the punch being 0.1 mm/s.

The test is carried out using a TA-XT21 texture analyser sold by the company Stable Micro System.

A curve of force F (in N) as a function of displacement d (in mm) is thus obtained, from which it is possible to determine the breaking point of the material (tensing agent) and the breaking energy W_{break} (J/m²) corresponding to the area under the curve F=f(d) at the breaking point F_{break} (N).

All the results obtained are given below:

Compositions

| | dᵣ (mm) | W_{break} (J/m²) |
|---|---|---|
| Example A: HYBRIDUR 875 (mosaic-effect | 0 | 0 |
| tensing agent according to the invention) | | |
| Example B: ELESERYL VGH8 | 0.4 | 40 |
| Example C: FLEXAN | 1.2 | 1600 |
| Example D: AVALURE UR 405 | 1.7 | 1400 |
| Example E: KYTAMER PCA | 1.5 | 3200 |

In particular, the mosaic-effect tensing agents according to the invention form a deposit characterized advantageously by a breaking energy of between 0 and 20 J/m² (preferably equal to 0) and a breaking deformation of between 0 and 0.2 mm in this mechanical strength test.

### Example 3

### Demonstration of the Effect of the Tensing Agents on the Reorganization of the Extracellular Matrix

### Principle of the Test

In order to define whether the application of tensing agents to the stratum corneum can induce modifications in terms of the organization of the extracellular matrix, 100 μl of tensing agents, respectively Hybridur 875 sold by the company Air Products (at 15% by weight in water) and an acrylic tensing agent (ethylenic copolymer as prepared in Example 1, at 7% by weight in water) were applied to a model of reconstructed skin of Episkin® type.

The model of reconstructed skin, consisting of human keratinocytes deposited on a substrate, commonly a dermis equivalent, and cultured under conditions such that they enter into a differentiation programme resulting in the formation of an epidermis equivalent, can be prepared according to the protocol described in Asselineau et al. (1987, Models in dermato., vol. 111, Ed. Lowe & Maibach, 1-7).

The Hybridur 875 sold by Air Products is prepared in accordance with the description in patents U.S. Pat. No. 5,977,215 and U.S. Pat. No. 5,521,246.

The effect of these tensing agents on the dermis is observed after 2 h, 24 h and 48 h of application.

The analyses are carried out by means of two complementary imaging techniques: optical microscopy (multiphoton microscopy and transmission optical microscopy on semi-thin sections) and electron microscopy (scanning electron microscopy).

Multiphoton microscopy makes it possible to define rapidly, and without prior preparation of the samples, the active agents which will exhibit an activity on the extracellular matrix, and over what timescale.

Three-dimensional resolution thereof makes it possible to determine the depth to which the mechanical stimulation will result in dermal modifications.

Once this first observation has been made, the samples are then analysed by scanning electron microscopy, which allows a better resolution and thus makes it possible to individualize the collagen fibrils.

The extracellular matrix modifications observed are related to modifications in the fibroblasts by virtue of studies by transmission optical microscopy on semi-thin sections.

### Results:

The electron and photon microscopy observations show, as represented in FIG. 3, that the tensing agents tested induce a reorganization of the collagen fibrils in the dermis after 48 h of application and also an extension and an increase in the number of the fibroblasts.

The collagen fibrils of the extracellular matrix associate with one another in a fibrillar structure in order to form networks that are more dense. This new organization can be linked to the synthesis of proteoglycans such as decorin or lumican, which are known to associate with several collagen molecules and thus group them together in a well-organized network.

The mechanical solicitations induced by the tensing agents thus stimulated the fibroblasts, resulting in a reorganization of their cytoskeleton, this taking place from 48 h after application onwards, and therefore over very short kinetics.

### Example 4

### Effect of the Combination of a Saccharide Compound which Increases the Expression of Integrins and a Tensing Agent

The effect of the combination of a saccharide compound which increases the expression of integrins (for example: C-glycoside derivative such as C-β-D-xylopyranoside-2-hydroxypropan-2-one) and a tensing agent, on the differential expression of genes involved in skin homeostasis, in particular certain cytokines such as TGFβ or matrix molecules (collagens and procollagen 1), was evaluated by RT-Q-PCR and/or by means of an ELISA assay on Episkin reconstructed epidermes or skin, compared to the effect of the C-glycoside derivative and of the tensing agent taken alone.

The EPISKIN® reconstructed epidermes used were obtained at D15. They were placed in a maintenance medium for 8 hours. They were then transferred into a DMEM/Ham F12 medium free from EGF, pituitary extract and foetal calf serum. The epidermes were equilibrated in this medium for 24 hours.

The EPISKINs® were then pretreated for 4 h and 24 h with 2 μg/ml of C-glycoside derivative at the surface of the keratinocytes. After this pre-treatment, one hundred microlitres of an aqueous dispersion of Hybridur 875 polymer sold by the company Air Products (at 15% by weight in water) was applied to the EPISKINs® in this culture medium and left in contact with the epidermes for 24 hours in a chamber thermostated at 37° C. and 40% relative humidity.

At the end of this period, the epidermes were removed and extracted for the RT-Q-PCR studies.

Other EPISKINs® were treated under the same conditions, but respectively with Hybridur 875 alone or C-glycoside derivative alone.

1) The effect of the products to be tested on the expression of the selected markers was evaluated by RT-Q-PCR carried out using the total RNA extracted from the epidermes, according to the following protocol:

The first step consists in carrying out a reverse transcription reaction.

This step requires a pre-treatment of the total RNA in order to remove traces of potentially contaminating DNA by treatment with the DNA-free system (Ambion). The reverse transcription of the mRNA to cDNA is carried out in the presence of the oligo(dT) primer and the Superscript II enzyme (Gibco).

The PCR (polymerase chain reaction) reactions were carried out by quantitative PCR with the “Light Cycler” system (Roche Molecular Systems Inc.) and according to the supplier's recommendations. This analytical system makes it possible to carry out rapid and efficient PCR reactions, with a prior setting up of the analytical conditions for the various primers. It is made up of two main components:
a thermocycler optimized through the use of glass capillaries and extremely rapid heat transfers;
a fluorimeter: which makes it possible to continuously measure the intensity of fluorescence incorporated into the DNA (detection of 521 nm).

The reaction mixture (10 μl final volume) introduced into capillaries for each sample is the following:
2.5 μl of cDNA diluted to 1/10,
primers for the various markers used,
reaction mixture (Roche) containing the taq DNA polymerase enzyme, the SYBR and Green I label (fluorophore which intercollates into the double-stranded DNA, during the extension step) and MgCl₂.

The incorporation of fluorescence into the amplified DNA is measured continuously over the course of the PCR cycles. This system makes it possible to obtain curves of the measurement of the fluorescence as a function of PCR cycles and thus to evaluate a relative expression value for each marker. The number of cycles is determined from the “exit” points of the fluorescence curves. For the same marker analysed, the later a sample exits (high cycle number), the lower the initial number of copies of the mRNA. The RE (relative expression) value is expressed in arbitrary units according to the following formula: (½ number of cycles)×10⁶.

2) The effect of the products to be tested on the expression of the selected markers (TGFβ and procollagen) can also be evaluated by ELISA assay. Briefly, after incubation, the culture media are sampled and the assay is carried out on a sample of medium using the specific assay kits from the supplier Bio-Whittaker.

### Example 5

### Cosmetic Compositions and Kits

A—Compositions Containing the Ethylenic Copolymer According to Example 1

Oil-in-water emulsion

| | |
|---|---|
| Phase A | |
| Glyceryl stearate (and) PEG-100 stearate (ARLACEL 165FL): | 2.00 g |
| Dimyristyl tartrate (and) cetearyl alcohol (and) C12-15 | 1.50 g |
| pareth-7 (and) PPG-25 laureth-25 (Cosmacol PSE): | |
| Cyclohexasiloxane: | 10.00 g |
| Stearyl alcohol: | 1.00 g |
| Phase B | |
| Water: | 41.5 g |
| Preserving agents: | 0.75 g |
| Pentasodium ethylenediaminetetramethylene phosphate: | 0.05 g |
| Ammonium polyacryldimethyltauramide | 0.40 g |
| (HOSTACERIN AMPS): | |
| C-β-D-xylopyranoside-2-hydroxy-propane | 1 g |
| Phase C | |
| Ethylenic copolymer according to Example 1 | 40.90 g |
| (dispersion at 7% in water): | |

### Procedure:

Phase B is heated to approximately 75° C. and the ammonium polyacryldimethyl-tauramide is incorporated therein; the mixture is agitated until a homogeneous gel is obtained;
phase A is heated to approximately 75° C.;
the emulsion is prepared by incorporating phase A into phase B;
at 40-45° C., phase C is incorporated and the agitation is continued until complete cooling.

Water-in-oil emulsion

| | | | |
|---|---|---|---|
| A | Polymethyl cetyldimethylmethylsiloxane oxyethylene | 1.5 | g |
| | Polyglyceryl isostearate | 0.5 | g |
| | Isohexadecane | 4 | g |
| | Squalane | 1.85 | g |
| | Dimethicone | 2.05 | g |
| | Apricot kernel oil | 1.1 | g |
| | Cyclopentasiloxane | 9 | g |
| | Propylparaben | 0.15 | g |
| B | Water | 29.2 | g |
| | Propylene glycol | 3 | g |
| | Magnesium sulphate | 1.75 | g |
| | Methylparaben | 0.2 | g |
| | Preserving agent | 0.3 | g |
| | Xylotetraose | 5 | g |
| C | Ethylenic copolymer prepared according to Example 1 | 40.9 | g |
| | (dispersion at 7% in water) | | |
| D | Nylon 12 | 3 | g |

### Procedure:

phase A and phase B are homogenized separately, at ambient temperature with agitation;
the emulsion is prepared by incorporating phase B into phase A;
phases C and D are incorporated with agitation.

Serum

| | | | |
|---|---|---|---|
| A | Water | 46.45 | g |
| | Ammonium polyacryldimethyltauramide | 2.00 | g |
| | (HOSTACERIN AMPS) | | |
| | Preserving agents | 0.85 | g |
| | C-β-D-xylopyranoside-2-hydroxy-propane | 5 | g |
| B | Ethylenic copolymer prepared according to Example 1 | 46.70 | g |
| | (dispersion at 7% in water) | | |

According to one alternative, the ethylenic copolymer is formulated in a separate composition for the preparation of a skincare kit.

B—Compositions Containing Hybridur 875

Hybridur 875 is sold by Air Products and is prepared in accordance with the description in patents U.S. Pat. No. 5,977,215 and U.S. Pat. No. 5,521,246.

Oil-in-water emulsion

| | | |
|---|---|---|
| A | Glyceryl stearate (and) PEG-100 stearate | 2.00 g |
| | (ARLACEL 165FL): | |
| | Dimyristyl tartrate (and) cetearyl alcohol (and) C12-15 | 1.50 g |
| | pareth-7 (and) PPG-25 laureth-25 (Cosmacol PSE): | |
| | Cyclohexasiloxane: | 10.00 g |
| | Stearyl alcohol: | 100 g |
| B | Water: | 66.10 g |
| | Preserving agents: | 0.75 g |
| | Pentasodium ethylenediaminetetramethylene phosphate: | 0.05 g |
| | Ammonium polyacryldimethyltauramide | 0.40 g |
| | (HOSTACERIN AMPS): | |
| | Xanthan gum (RHODICARE S): | 0.20 g |
| | C-β-D-xylopyranoside-2-hydroxy-propane | 100 g |
| C | Hybridur 875 | 17.00 g |

### Procedure:

Phase B is heated to approximately 75° C. and the ammonium polyacryldimethyl-tauramide is incorporated therein; the mixture is agitated until a homogeneous gel is obtained;
phase A is heated to approximately 75° C.;
the emulsion is prepared by incorporating phase A into phase B;
at 40-45° C., phase C is incorporated and the agitation is continued until complete cooling.

W/O/W triple emulsion

| | |
|---|---|
| Primary emulsion (A): | |
| Water: | 58.20 g |
| Polyglyceryl-4 isostearate, hexyl laurate and | 3.50 g |
| cetyl PEG/PPG 10/1 dimethicone (ABILWE09): | |
| Cyclopentasiloxane: | 16.50 g |
| Dimethicone: | 4.00 g |
| Hybridur 875 | 17.00 g |
| Magnesium sulphate | 0.80 g |
| Multiple emulsion: | |
| Primary emulsion (A): | 22.50 g |
| Cyclopentasiloxane: | 3.50 g |
| Apricot kernel oil: | 4.00 g |
| Water: | 65.05 g |
| Preserving agents | 1.00 g |
| Pentasodium ethylenediaminetetramethylene phosphonate: | 0.05 g |
| Alkyl acrylate copolymer (PEMULEN TR1): | 0.60 g |
| Sodium hydroxide: | 0.30 g |
| C-β-D-xylopyranoside-2-hydroxy-propane | 0.50 g |

### Procedure:

### Preparation of the Primary Emulsion:

The polyglyceryl-4 isostearate, hexyl laurate, cetyl PEG/PPG 10/1 dimethicone, cyclopentasiloxane and dimethicone are homogenized at ambient temperature with agitation. The water and the Hybridur 875 are incorporated slowly with vigorous agitation.

### Preparation of the Triple Emulsion:

The alkyl acrylate copolymer, the preserving agents and the sequestering agent (pentasodium ethylenediaminetetramethylene phosphonate) are dispersed at ambient temperature and with agitation. Swelling is allowed to occur for approximately 45 minutes with agitation, followed by neutralization with sodium hydroxide. The primary emulsion is diluted with the cyclopentasiloxane and the apricot kernel oil, and then this mixture is slowly incorporated into the aqueous phase with agitation.

W/O emulsion

| | | | |
|---|---|---|---|
| A | Polymethyl cetyldimethylmethylsiloxane oxyethylene | 1.5 | g |
| | (ABIL EM90) | | |
| | Polyglyceryl isostearate (ISOLAN Gl34) | 0.5 | g |
| | Isohexadecane | 4 | g |
| | Squalane | 1.85 | g |
| | Dimethicone | 2.05 | g |
| | Apricot kernel oil | 1.1 | g |
| | Cyclopentasiloxane | 9 | g |
| | Propylparaben | 0.15 | g |
| B | Water | 54.10 | g |
| | Propylene glycol | 3 | g |
| | Magnesium sulphate | 1.75 | g |
| | Methylparaben | 0.2 | g |
| | Preserving agent | 0.3 | g |
| | Aqueous extract of arabinoxylan (COHELISS © SILAB) | 1 | g |
| C | Hybridur 875 | 17.00 | g |
| D | Nylon 12 | 3 | g |

### Procedure:

phase A and phase B are homogenized separately, at ambient temperature with agitation;
the emulsion is prepared by incorporating phase B into phase A;
phases C and D are incorporated with agitation.

Serum

| | | | |
|---|---|---|---|
| A | Water | 79.65 | g |
| | Ammonium polyacryldimethyltauramide | 2.00 | g |
| | (HOSTACERIN AMPS) | | |
| | Preserving agents | 0.85 | g |
| | Aqueous extract of arabinoxylan (COHELISS © SILAB) | 5 | g |
| B | Hybridur 875 | 17.00 | g |

According to one alternative, the Hybridur 875 is formulated in a separate composition for the preparation of a skincare kit.

### Example 6

### Effect of the Combination of a Saccharide Compound which Increases the Expression of Integrins and of a Tensing Agent Intended to Apply and/or Maintain a Tension to and/or on the Skin

a) Device Intended to Maintain a Tension

The effect of the combination of a saccharide compound which increases the expression of integrins (for example: arabinoxylan or C-glycoside derivative such as C-β-D-xylopyranoside-2-hydroxypropan-2-one) and a device for maintaining a tension (for example: patch), on the differential expression of genes involved in skin homeostasis (for example: TGFβ, keratin 19 and HSP90A), was evaluated by RT-Q-PCR on EPISKIN® reconstructed epidermes, compared to the effect of the peptide and of the patch taken alone.

A patch having an elastic modulus of greater than 500 MPa was used. Patches with an elastic modulus of 500 MPa, 1000 MPa, 1500 MPa and 2000 MPa were tested.

The EPISKIN® reconstructed epidermes used were obtained at D15. They were placed in a maintenance medium for 8 hours. They were then transferred into a DMEM/Ham F12 medium free from EGF, pituitary extract and foetal calf serum. The epidermes were equilibrated in this medium for 24 hours.

The EPISKINs® were then pretreated for 4 h and 24 h with 2 μg/ml of C-glycoside derivative such as C-β-D-xylopyranoside-n-propan-2-one at the surface of the keratinocytes. After this pre-treatment, a patch-type mechanical device was applied to the EPISKINs® already subjected to a tension, in this culture medium, and left in contact with the epidermes for 24 hours in a chamber thermostated at 37° C. and 40% relative humidity.

At the end of this period, the epidermes were removed and extracted for the RT-Q-PCR studies.

Other EPISKINs® were treated under the same conditions, but respectively with the patch alone or arabinoxylan or the C-glycoside derivative alone.

The effect of the products to be tested, on the expression of the selected markers was evaluated by RT-Q-PCR carried out using the total RNA extracted from the epidermes, according to the following protocol:

The first step consists in carrying out a reverse transcription reaction.

This step requires a pre-treatment of the total RNA in order to remove traces of potentially contaminating DNA by treatment with the DNA-free system (Ambion). The reverse transcription of the mRNA to cDNA is carried out in the presence of the oligo(dT) primer and the Superscript II enzyme (Gibco).

The PCR (polymerase chain reaction) reactions were carried out by quantitative PCR with the “Light Cycler” system (Roche Molecular Systems Inc.) and according to the supplier's recommendations. This analytical system makes it possible to carry out rapid and efficient PCR reactions, with a prior setting up of the analytical conditions for the various primers. It is made up of two main components:
a thermocycler optimized through the use of glass capillaries and extremely rapid heat transfers;
a fluorimeter: which makes it possible to continuously measure the intensity of fluorescence incorporated into the DNA (detection of 521 nm).

The reaction mixture (10 μl final volume) introduced into capillaries for each sample is the following:
2.5 μl of cDNA diluted to 1/10e,
primers for the various markers used,
reaction mixture (Roche) containing the taq DNA polymerase enzyme, the SYBR and Green I marker (fluorophore which intercollates into the double-stranded DNA, during the extension step) and MgCl₂.

The incorporation of fluorescence into the amplified DNA is measured continuously over the course of the PCR cycles. This system makes it possible to obtain curves of measurement of the fluorescence as a function of PCR cycles and thus to evaluate a relative expression value for each marker. The number of cycles is determined from the “exit” points of the fluorescence curves. For the same marker analysed, the later a sample exits (high cycle number), the lower the initial number of copies of the mRNA. The RE (relative expression) value is expressed in arbitrary units according to the following formula: (½ number of cycles)×10⁶.

b) Device Intended to Apply a Tension and a Pressure to the Skin

According to an alternative embodiment, as a replacement for the patch used in a), a device of the TA-XT2i texture analyser type sold by the company Stable Micro System is used. This analyser is suitable for application to Episkin® and has a hemispherical head.

A tension and a compression (pressure) can thus be applied to Episkin® under various conditions ranging from 5 MPa to 90 MPa, in particular 5 MPa, 10 MPa, 20 MPa, 50 MPa and 90 MPa.

The same procedure as that described in a) is followed.

### Example 7

### Compositions and Application Routine

### Example A

### Cosmetic Composition—Oil-In-Water Emulsion

| | |
|---|---|
| A | |
| Glyceryl stearate (and) PEG-100 stearate (ARLACEL 165FL): | 2.00 g |
| Dimyristyl tartrate (and) cetearyl alcohol (and) C12-15 | 1.50 g |
| pareth-7 (and) PPG-25 laureth-25 (Cosmacol PSE): | |
| Cyclohexasiloxane: | 10.00 g |
| Stearyl alcohol: | 1.00 g |
| B | |
| Preserving agents: | 0.75 g |
| Pentasodium ethylenediaminetetramethylene phosphate: | 0.05 g |
| Ammonium polyacryldimethyltauramide | 0.40 g |
| (HOSTACERIN AMPS): | |
| C-β-D-xylopyranoside-2-hydroxy-propane | 1.80 g |
| Water: | qsp |

### Procedure:

Phase B is heated to approximately 75° C. and the ammonium polyacryldimethyltauramide is incorporated therein; the mixture is agitated until a homogeneous gel is obtained;
phase A is heated to approximately 75° C.;
the emulsion is prepared by incorporating phase A into phase B.

The composition is applied to the skin of the face and/or body and the area of skin over which said composition was applied is massaged with a manual massage instrument of the Environ Cosmetic Roll-Cit® type. The duration of the massage can range from 1 minute to several minutes in order to optimize the desired effect on skin homeostasis and/or the mechanical properties of the skin.

### Example B

### Cosmetic Composition in the form of a W/O/W Emulsion

| | |
|---|---|
| Primary emulsion (A): | |
| Polyglyceryl-4 isostearate, hexyl laurate and cetyl PEG/PPG | 3.50 g |
| 10/1 dimethicone: | |
| Cyclopentasiloxane: | 16.50 g |
| Dimethicone: | 4.00 g |
| Magnesium sulphate: | 0.80 g |
| Water: | qsp |
| Multiple emulsion: | |
| Primary emulsion (A): | 22.50 g |
| Cyclopentasiloxane: | 3.50 g |
| Apricot kernel oil: | 4.00 g |
| Water: | 65.05 g |
| Preserving agents | 1.00 g |
| Pentasodium ethylenediaminetetramethylene phosphonate: | 0.05 g |
| Alkyl acrylate copolymer: | 0.60 g |
| Sodium hydroxide: | 0.30 g |
| C-β-D-xylopyranoside-2-hydroxy-propane | 3 g |

### Procedure:

### Preparation of the Primary Emulsion:

The polyglyceryl-4 isostearate, hexyl laurate, cetyl PEG/PPG 10/1 dimethicone, cyclopentasiloxane and dimethicone are homogenized at ambient temperature with agitation. The water is slowly incorporated with vigorous agitation.

### Preparation of the Triple Emulsion:

The alkyl acrylate copolymer, the preserving agents and the sequestering agent (pentasodium ethylenediaminetetramethylene phosphonate) are dispersed at ambient temperature and with agitation. Swelling is allowed to occur for approximately 45 minutes with agitation, followed by neutralization with sodium hydroxide. The primary emulsion is diluted with the cyclopentasiloxane and the apricot kernel oil, and then this mixture is slowly incorporated into the aqueous phase with agitation.

This composition is applied to the areas of the body where the skin is soft and/or flaccid, in particular the stomach and/or the thighs. A massage is then performed using a device of the Lift6® type for a period ranging from 10 to 30 minutes in order to optimize the desired effect on the elasticity and/or the firmness of the skin.

### Example C

### Cosmetic Composition in the Form of a W/O Emulsion

| A | Polymethyl cetyldimethylmethylsiloxane oxyethylene | 1.5 | g |
|---|---|---|---|
| | Polyglyceryl isostearate | 0.5 | g |
| | Isohexadecane | 4 | g |
| | Squalane | 1.85 | g |
| | Dimethicone | 2.05 | g |
| | Apricot kernel oil | 1.1 | g |
| | Cyclopentasiloxane | 9 | g |
| | Propylparaben | 0.15 | g |
| B | Propylene glycol | 3 | g |
| | Magnesium sulphate | 1.75 | g |
| | Methylparaben | 0.2 | g |
| | Preserving agent | 0.3 | g |
| | Xylotetraose | 1.5 | g |
| | Water | qsp | |
| C | Nylon 12 | 3 | g |

### Procedure:

Phase A and phase B are homogenized separately, at ambient temperature with agitation;
the emulsion is prepared by incorporating phase B into phase A;
phase C is incorporated with agitation.

### Example D

### Serum

| | | |
|---|---|---|
| A | Ammonium polyacryldimethyltauramide | 2.00 g |
| | (HOSTACERIN AMPS) | |
| | Preserving agents | 0.85 g |
| | Aqueous extract of arabinoxylan (COHELISS © SILAB) | 5 g |
| | Water | qsp |

The area of skin to be treated is massaged, in the morning, with a manual massage instrument of the Environ Cosmetic Roll-Cit® type. The duration of the massage can range from 1 minute to several minutes in order to optimize the desired effect on skin homeostasis and/or the mechanical properties of the skin. In addition, the composition described above is applied in the evening.

1-45. (canceled)

46. A composition for topical application to the skin, comprising, in a physiologically acceptable medium, at least one tensing agent and at least one saccharide compound which increases the expression of mechanoreceptors in skin cells, said saccharide compound being different from the tensing agent.

47. The composition according to claim 46, characterized in that the saccharide compound increases the expression of integrins in skin cells.

48. The composition according to claim 46, characterized in that the tensing agent is chosen from synthetic polymers, plant or animal proteins, polysaccharides of natural origin which may or may not be in the form of microgels, mixed silicates, colloidal particles of inorganic fillers, and mixtures thereof.

49. The composition according to claim 48, characterized in that the tensing agent is chosen from silicone polymers comprising a polysiloxane backbone grafted with non-silicone organic monomers.

50. The composition according to claim 48, characterized in that the tensing agent is chosen from colloidal particles of silica.

51. The composition according to claim 48, characterized in that the tensing agent is chosen from acrylic copolymers consisting of (a) 70% to 90% by weight of the copolymer of at least one alkyl acrylate and/or at least one alkyl methacrylate and/or styrene and (b) 10% to 30% by weight of a copolymer of at least one ionic hydrophilic monomer.

52. The composition according to claim 48, characterized in that the tensing agent is an ethylenic copolymer.

53. The composition according to claim 48, characterized in that the tensing agent is a polymer of interpenetrating polymer network type.

54. The composition according to claim 53, characterized in that the interpenetrating polymer network comprises a polyurethane polymer and an acrylic polymer.

55. The composition according to claim 46, characterized in that the tensing agent is present in the composition in an amount ranging from 0.1% to 30% by weight relative to the total weight of the composition.

56. The composition according to claim 47, characterized in that the saccharide compound which increases the expression of integrins in skin cells is chosen from monosaccharides, polysaccharides, and derivatives thereof.

57. The composition according to claim 56, characterized in that the polysaccharide is a xylan derivative.

58. The composition according to claim 56, characterized in that the monosaccharide or polysaccharide derivatives are C-glycoside compounds of general formula (I) 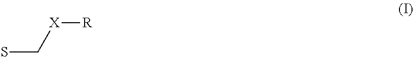
in which:
X represents a radical chosen from the groups: 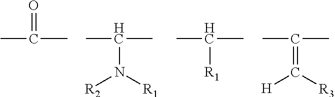
with R₁, R₂ and R₃ representing, independently of one another, a hydrogen atom, an OH group or a radical R, with R as defined below,
R represents:
a saturated or unsaturated, linear, branched or cyclic C₁ to C₂₀ alkyl radical,
a saturated or unsaturated, linear, branched or cyclic C₁ to C₂₀ polyfluoroalkyl or perfluoroalkyl radical, or
a C₅ to C₂₀ aryl, in particular phenyl or alkylaryl radical, in particular benzyl,
it being possible for the hydrocarbon-based chain constituting said radicals to be, where appropriate, interrupted with 1, 2, 3 or more heteroatoms chosen from:
oxygen,
sulphur,
nitrogen, and
silicon
and to be optionally substituted with at least one radical chosen from:
—OR₄,
—SR₄,
—NR₄R₅,
—COOR₄,
—CONHR₄,
CN,
a halogen atom,
a C₁ to C₆ polyfluoroalkyl or perfluoroalkyl radical,
a C₃ to C₈ cycloalkyl or heterocycloalkyl radical, and
a C₅ to C₁₈ aryl radical, optionally substituted,
with R₄ and R₅ possibly representing, independently of one another, a hydrogen atom, a hydroxyl radical or a saturated or unsaturated, linear or branched C₁ or C₃₀, in particular C₁ to C₁₂, alkyl, acyl, perfluoroalkyl or polyfluoroalkyl radical,
S represents a monosaccharide or a polysaccharide containing up to 20 sugar units, in particular up to 6 sugar units, in pyranose and/or furanose form and of the L and/or D series, it being possible for said monosaccharide or polysaccharide to be substituted with a (CH₂)—OR₆ radical, with R₆ representing a hydrogen atom or a C₁ to C₆ alkyl radical, with a hydroxyl group and/or with an O-glycoside radical, and having at least one free hydroxyl function and/or an optionally protected amine function, and
the S—C bond represents a bond of C-anomeric nature, or a salt or isomer thereof.

59. The composition according to claim 46, characterized in that the saccharide compound which increases the expression of mechanoreceptors is present in the composition in an amount ranging from 0.00001% to 30% by weight relative to the total weight of the composition.

60. A skin care kit comprising at least:
a first composition comprising, in a physiologically acceptable medium, at least one saccharide compound which increases the expression of mechanoreceptors in skin cells; and
a second composition comprising, in a physiologically acceptable medium, at least one tensing agent.

61. The kit according to claim 60, characterized in that it also comprises a tensing device intended to apply and/or maintain, in a controlled manner, mechanical stresses to and/or on the skin.

62. A process for the cosmetic treatment of the skin, through the application to the skin, via the use of at least one composition, of at least one saccharide compound which increases the expression of mechanoreceptors in skin cells and of at least one tensing agent.
